(19) United States
(12) Patent Application Publication
(10) Pub. No.: US 2024/0423994 A1
(43) Pub. Date: Dec. 26, 2024

(54) TARGETING IMMUNE SUPPRESSION AFTER ATR INHIBITION

(71) Applicant: BROWN UNIVERSITY, Providence, RI (US)

(72) Inventors: Wafik S. El-Deiry, Barrington, RI (US); Maryam Ghandali, Providence, RI (US)

(21) Appl. No.: 18/677,223

(22) Filed: May 29, 2024

Related U.S. Application Data
(60) Provisional application No. 63/504,891, filed on May 30, 2023.

(51) Int. Cl. A61K 31/5355; A61K 31/506; A61K 39/00; C07K 16/22

(52) U.S. Cl. CPC A61K 31/5355; A61K 31/506; C07K 16/22; A61K 2039/505; C07K 2317/565

(57) ABSTRACT

The present disclosure provides, for instance, a method of inhibiting resistance to a DDR inhibitor in a subject, the method comprising administering to the subject a GDF15 inhibitor, thereby inhibiting resistance to the DDR inhibitor in the subject. The disclosure also provides combination therapies for cancer, comprising co-administration of a DDR inhibitor and a GDF15 inhibitor.

### CROSS REFERENCE TO RELATED APPLICATIONS

This application claims the benefit of U.S. Provisional Patent Application No. 63/504,891, filed May 30, 2023. The contents of the aforementioned application are hereby incorporated by reference in their entirety.

### SEQUENCE LISTING

The instant application contains a Sequence Listing which has been submitted electronically in XML file format and is hereby incorporated by reference in its entirety. Said XML copy, created on May 28, 2024, is named 0312021_00036_SL.xml and is 115,836 bytes in size.

### BACKGROUND

The Ataxia telangiectasia and Rad3-related protein (ATR) is a human serine/threonine protein kinase involved in DNA repair. ATR is activated upon detection of single stranded DNA breaks, and activates the DNA damage checkpoint.

DNA damage and repair (DDR) inhibitors, such as ATR inhibitors, show promise in cancer therapy. However, a problem in cancer therapy is the emergence of resistance to these inhibitors. Cancer progression due to evasion of anti-cancer immune response contributes to cancer progression and to morbidity and mortality in patients with cancer. There is a need in the art for new therapies that avoid or reduce resistance to DDR inhibitors.

### SUMMARY OF THE INVENTION

In some aspects, the present disclosure provides a method of treating cancer in a subject, the method comprising: administering to the subject an effective amount of a DNA damage and repair (DDR) inhibitor, wherein the subject receives, in combination, a GDF15 inhibitor, thereby treating the cancer in the subject. In some embodiments, the method comprises administering the GDF15 to the subject. In some aspects, the present disclosure provides a DNA damage and repair (DDR) inhibitor for use, in combination with a GDF15 inhibitor, in the treatment of a cancer in a subject. In some aspects, the present disclosure provides the use of a DNA damage and repair (DDR) inhibitor, in combination with a GDF15 inhibitor, for the manufacture of a medicament for the treatment of a cancer in a subject.

In some aspects, the present disclosure provides a method of treating cancer in a subject, the method comprising: administering to the subject an effective amount of a GDF15 inhibitor, wherein the subject receives, in combination, a DDR inhibitor, thereby treating the cancer in the subject. In some embodiments, the method comprises administering the DDR inhibitor to the subject. In some aspects, the present disclosure provides a GDF15 inhibitor for use, in combination with a DDR inhibitor, in the treatment of a cancer in a subject. In some aspects, the present disclosure provides the use of a GDF15 inhibitor, in combination with a DDR inhibitor, for the manufacture of a medicament for the treatment of a cancer in a subject.

In some aspects, the present disclosure provides a method of inhibiting resistance to a DDR inhibitor in a subject, the method comprising administering to the subject a GDF15 inhibitor, thereby inhibiting resistance to the DDR inhibitor in the subject. In some aspects, the present disclosure provides a GDF15 inhibitor for use in inhibiting resistance to a DDR inhibitor in a subject. In some aspects, the present disclosure provides the use of a GDF15 inhibitor for the manufacture of a medicament for inhibiting resistance to a DDR inhibitor in a subject.

In some aspects, the present disclosure provides a kit comprising: a DDR inhibitor or a nucleic acid encoding the DDR inhibitor; and a GDF15 inhibitor or a nucleic acid encoding the GDF15 inhibitor.

In some embodiments (e.g., of the methods, uses, and kits described herein) the DDR inhibitor comprises an ATR inhibitor or a PARP inhibitor. In some embodiments, the DDR inhibitor comprises a small molecule, an antibody molecule, a nucleic acid, or a polypeptide.

In some embodiments, the DDR inhibitor comprises ceralasertib, camonsertib, elimusertib, or berzosertib. In some embodiments, the DDR inhibitor comprises ceralasertib and is administered daily, e.g., at a daily dose of between 10 mg and 100 mg. e.g., between 20 mg and 60 mg. e.g., between 30 mg and 50 mg. In some embodiments, the DDR inhibitor comprises camonsertib and is administered daily, e.g., at a daily dose of between 50 mg and 300 mg, e.g., between 100 mg and 200 mg, e.g. between 150 mg and 170 mg, optionally wherein the DDR inhibitor is administered daily for 3 days. In some embodiments, the DDR inhibitor comprises elimusertib and is administered daily, e.g., at a daily dose of between 20 mg and 200 mg, e.g. between 50 mg and 120 mg, e.g. between 70 mg and 90 mg, optionally wherein the DDR inhibitor is administered daily for 3 days. In some embodiments, the DDR inhibitor comprises berzosertib and is administered, e.g. at a dose of between 100 mg/m² and 400 mg/m², e.g. between 150 mg/m² and 250 mg/m², e.g. between 200 mg/m² and 220 mg/m² per day.

In some embodiments, the GDF15 inhibitor comprises a small molecule, an antibody molecule, a nucleic acid, or a polypeptide. In some embodiments, the GDF15 inhibitor comprises an antibody molecule having a light chain complementary determining region 1 (LC CDR1), a light chain complementary determining region 2 (LC CDR2), a light chain complementary determining region 3 (LC CDR3), a heavy chain complementary determining region 1 (HC CDR1), a heavy chain complementary determining region 2 (HC CDR2), and a heavy chain complementary determining region 3 (HC CDR3) having amino acid sequences according to Table 1, Table 3, Table 4, or Table 6. In some embodiments, the GDF15 inhibitor comprises an antibody molecule having a light chain variable region (VL) comprising an amino acid sequence according to Table 2, Table 5, or Table 7, or a sequence having at least 90%, 95%, 97%, 98%, or 99% identity thereto. In some embodiments, the GDF15 inhibitor comprises an antibody molecule having a heavy chain variable region (VH) comprising an amino acid sequence according to Table 2. Table 5, or Table 7, or a sequence having at least 90%, 95%, 97%, 98%, or 99% identity thereto. In some embodiments, the GDF15 inhibitor comprises AV-380, ponsegromab, or visugromab.

In some embodiments, the DDR inhibitor is administered orally, parenterally, intravenously, or topically. In some embodiments, the GDF15 inhibitor is administered intravenously, orally, parenterally, or topically. In some embodiments: the DDR inhibitor and the GDF15 inhibitor are administered simultaneously; the DDR inhibitor is administered before the GDF15 inhibitor; or the GDF15 inhibitor is administered before the DDR inhibitor.

In some embodiments, the subject comprises a mutation in BRCA1 or BRCA2. In some embodiments, the subject is resistant to or refractory to a DDR inhibitor, e.g., wherein the subject is resistant to or refractory to one or more of ceralasertib, camonsertib, elimusertib, or berzosertib. In some embodiments, the subject is, or is identified as being, at risk of developing resistance to, or at risk of being refractory to, a DDR inhibitor. In some embodiments, following administration of the GDF15 inhibitor, the subject does not develop resistance to the DDR inhibitor, or wherein resistance to the DDR inhibitor is delayed or decreased. In some embodiments, following administration of the GDF15 inhibitor, the subject displays increased (e.g., fully restored) sensitivity to the DDR inhibitor. In some embodiments, the subject has a solid tumor. In some embodiments, the cancer is chosen from breast cancer, ovarian cancer, pancreatic cancer, fallopian tube cancer, primary peritoneal cancer, or prostate cancer. In some embodiments, the subject has a liquid tumor. In some embodiments, the cancer is a leukemia or lymphoma. In some embodiments, the subject is a human. In some embodiments, the DDR inhibitor and the GDF15 inhibitor are administered by the same entity or by different entities.

The present disclosure contemplates all combinations of the aspects and embodiments disclosed herein.

### DETAILED DESCRIPTION OF THE INVENTION

The present disclosure provides, for example, methods of targeting cancer therapy resistance to ATR inhibition by blocking an immune suppressive mechanism involving GDF15 within the tumor microenvironment. The disclosure also provides a previously undescribed mechanism of evasion of anti-tumor immunity whose targeting would be helpful for patients receiving anti-cancer therapy with DDR inhibitors such as ATR inhibitory drugs.

Accordingly, the present disclosure provides, for instance, combination therapies comprising a DDR inhibitor and a GDF15 inhibitor. Exemplary DDR inhibitors and GDF15 inhibitors are described herein.

In the following description, for an explanation, numerous specific details provide a thorough understanding of the compositions and methods disclosed herein. However, it may be evident that the compositions and methods may be practiced without these specific details. Aspects, modes, embodiments, variations, and features of the compositions and methods are described below in various levels of detail to provide a substantial understanding of the present disclosure.

### Definitions

For convenience, the meaning of some terms and phrases used in the specification, examples, and appended claims, are listed below. Unless otherwise defined, all technical and scientific terms have the same meaning as commonly understood by a person having ordinary skill in the biomedical art to which this invention belongs. A term's meaning provided in this specification shall prevail if any apparent discrepancy arises between the meaning of a definition provided in this specification and the term's use in the biomedical art.

The singular forms a, an, and the like include plural referents unless the context dictates otherwise. For example, a reference to a cell comprises a combination of two or more cells.

As used herein, the term “antibody molecule” refers to a naturally occurring antibody, an engineered antibody, or a fragment thereof. In some embodiments, an antibody molecule is an antigen binding portion of a naturally occurring antibody or an engineered antibody. In some embodiments, an antibody molecule includes an antibody or an antigen-binding fragments thereof (e.g., Fab, Fab′, F(ab′)2, Fv fragments, scFv antibody fragments, disulfide-linked Fvs (sdFv), Fd fragments consisting of the VH and CH1 domains, linear antibodies, single domain antibodies such as sdAb (either VL or VH), nanobodies, or camelid VHH domains). an antigen-binding fibronectin type III (Fn3) scaffold such as a fibronectin polypeptide minibody, a ligand, a cytokine, a chemokine, or a T cell receptor (TCR). In some embodiments, an antibody molecule is a humanized antibody molecule. In some embodiments. an antibody molecule is an intact IgA, IgG, IgE or IgM antibody. In some embodiments, an antibody molecule is a bi-or multi-specific antibody (e.g., Zybodies®), etc). In some embodiments, antibody molecules are antibody fragments such as Fab fragments, Fab′ fragments, F(ab′)β fragments, Fd′ fragments, Fd fragments, isolated CDRs or sets thereof. In some embodiments, an antibody molecule is a single chain Fv (scFv), a polypeptide-Fc fusion, a single domain antibody (e.g., shark single domain antibodies such as IgNAR or fragments thereof), or a cameloid antibody. In some embodiments, antibody molecules are masked antibodies (e.g., Probodies®), Small Modular ImmunoPharmaceuticals (“SMIPsTM”), single chain or Tandem diabodies (TandAb®), VHHs; Anticalins®, Nanobodies®, minibodies, BiTE®s, ankyrin repeat proteins or DARPINS®, Avimers®, DARTs, TCR-like antibodies, Adnectins®, Affilins®, Trans-bodies®, Affibodies®, TrimerX®, MicroProteins, Fynomers®, Centyrins®, or KALBITOR®s.

As used herein, the term “ATR inhibitor” refers to an agent that specifically binds and inhibits activity of an Ataxia telangiectasia and Rad3 related (ATR) protein, or an agent that specifically reduces expression of the ATR protein. In some embodiments, the agent comprises a molecule or a complex. In some embodiments, the agent that specifically reduces expression of the ATR protein comprises a nucleic acid that binds ATR mRNA and reduces the level or translation of the ATR mRNA.

As used herein, the term “cancer” refers to a disease characterized by the rapid and uncontrolled growth of aberrant cells. In some embodiments, cancer cells can spread locally or through the bloodstream and lymphatic system to other parts of the body. In some embodiments, the cancer is a primary cancer. In some embodiments, the cancer is premalignant or malignant. In some embodiments the cancer is a solid tumor or a liquid tumor. In some embodiments, the cancer is actively proliferating. In some embodiments, the cancer comprises a dormant tumor or a micrometastasis.

As used herein, the terms “complementarity determining region” and “CDR” are interchangeable and refer to the sequences of amino acids within antibody variable regions which confer antigen specificity and binding affinity. In some embodiments, the precise amino acid sequence boundaries of a given CDR are determined using the method described by Kabat et al. (1991), “Sequences of Proteins of Immunological Interest,” 5th Ed. Public Health Service, National Institutes of Health, Bethesda, MD (“Kabat” numbering scheme). In some embodiments, the precise amino acid sequence boundaries of a given CDR are determined using the method described by Al-Lazikani et al., (1997) JMB 273, 927-948 (“Chothia” numbering scheme). In some embodiments, the precise amino acid sequence boundaries of a given CDR are determined using the method described by Lefranc, M.-P., The Immunologist, 7, 132-136 (1999) and Lefranc, M.-P. et al., Dev. Comp. Immunol., 27, 55-77 (2003) (“IMGT” numbering scheme).

As used herein, the term “combination” refers to either a fixed combination in one dosage unit form, or a combined administration where an agent described herein and a combination partner may be administered independently to a single subject at the same time or separately within time intervals. In some embodiments, these time intervals allow that the combination partners show a synergistic effect. The single components may be packaged in a kit or separately. In some embodiments, administration of two agents in combination includes treatment regimens in which the agents are not necessarily administered by the same route of administration or at the same time. The term “fixed combination” means that the therapeutic agents are both administered to a subject simultaneously in the form of a single entity or dosage.

As used herein, the term “comprising” means that other elements can also be present in addition to the defined elements presented. Using comprising indicates inclusion rather than limitation.

As used herein, the term “consisting essentially of” means the listed elements are required for a given embodiment. The term permits additional elements that do not materially affect the basic and functional characteristics of that embodiment of the invention.

As used herein, the term “consisting of” means compositions, methods, and respective components thereof, exclusive of any element not recited in that description of the embodiment.

As used herein, the term “DNA damage and repair (DDR) inhibitor” refers to an agent that specifically inhibits activity or reduces expression of a protein in a DDR pathway. In some embodiments, the agent comprises a molecule or a complex. In some embodiments, the agent that specifically reduces expression of the DDR protein comprises a nucleic acid that binds DDR mRNA and reduces the level or translation of the DDR mRNA. In some embodiments, the DDR inhibitor comprises an ATR inhibitor or a PARP inhibitor.

As used herein, the term “effective amount” refers to the amount sufficient to cause beneficial or desired results, for example, clinical results, and, as such, an “effective amount” depends upon the context in which it is being applied. Many ways are known in the biomedical art to determine the effective amount for an application. For example, pharmacological methods for dosage determination can be used in the therapeutic context. In therapeutic or prophylactic applications, the amount of a composition administered to the subject depends on the type and severity of the disease and the characteristics of the individual, such as general health, age, sex, body weight, tolerance to drugs, and on the degree, severity, and type of disease. Persons having ordinary skill in the biomedical art can determine appropriate dosages depending on these and other factors. In some embodiments, an effective amount results in inhibition of a target protein by at least 5%, 10%, 20%, 30%, 40%, 50%, 60%, 70%, 80%, 90%, or more. In some embodiments, an effective amount results in a reduction of the size of a tumor by at least 5%, 10%, 20%, 30%, 40%, 50%, 60%, 70%, 80%, 90%, or more.

As used herein, the term “expression” refers to the transcription or translation of a particular nucleic acid sequence driven by a promoter. In some embodiments, expression refers to the level of accumulation of an RNA. In some embodiments, expression refers to the accumulation of a protein. As used herein, the term “GDF15 inhibitor” refers to an agent that specifically binds and inhibits activity of a Growth/differentiation factor-15 (GDF15) protein, or an agent that specifically reduces expression of the GDF15 protein. In some embodiments, the agent comprises a molecule or a complex. In some embodiments the agent comprises an antibody molecule. In some embodiments, the agent that specifically reduces expression of the GDF15 protein comprises a nucleic acid that binds GDF15 mRNA and reduces the level or translation of the GDF15 mRNA.

As used herein, the term “inhibitor” refers to an agent that causes a decrease of a certain parameter. In some embodiments, the parameter is an activity of a given molecule, e.g. phosphorylation activity of ATR kinase. In some embodiments, the decrease is a decrease by at least 10% as compared to a reference level (e.g., the absence of a treatment or agent) and can include more significant decreases, for example, a decrease by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. Activities for the inhibitors can be determined using any suitable assay, for instance, an assay as described herein or an assay known in the art. In some embodiments, the inhibitor specifically binds a target molecule. In some embodiments, the inhibitor prevents the activity of a bound target molecule. In some embodiments, the inhibitor reduces expression of a given molecule.

As used herein, the term “nucleic acid” refers to a polymeric molecule incorporating units of ribonucleic acid, deoxyribonucleic acid, or an analog thereof. In some embodiments, the nucleic acid is in single stranded form. In some embodiments, the nucleic acid is in double stranded form. In some embodiments, the nucleic acid is genomic DNA, cDNA, or RNA (e.g. mRNA). In some embodiments, the nucleic acid contains analogues of natural nucleotides that have similar binding properties as the reference nucleic acid. In some embodiments, the nucleic acid containing analogues of natural nucleotides are metabolized in a manner similar to naturally occurring nucleotides.

As used herein, the term “or” refers to and/or. The term and/or as used in a phrase such as A and/or B herein includes both A and B; A or B; A (alone); and B (alone). Likewise, the term and/or as used in a phrase such as A, B, and/or C encompasses each embodiment: A, B, and C; A, B, or C; A or C; A or B; B or C; A and C; A and B; Band C; A (alone); B (alone); and C (alone).

As used herein, the term “PARP inhibitor” refers to an agent that specifically binds and inhibits activity of a poly ADP ribose polymerase (PARP) protein, or an agent that specifically reduces expression of the PARP protein. In some embodiments, the PARP protein is PARP1 or PARP2. In some embodiments, the PARP inhibitor inhibits more than one PARP protein. In some embodiments, the agent comprises a molecule or a complex. In some embodiments, the agent that specifically reduces expression of the PARP protein comprises a nucleic acid that binds PARP mRNA and reduces the level or translation of the PARP mRNA.

As used herein, the terms “peptide,” “polypeptide,” and “protein” are used interchangeably, and refer to a molecule comprised of two or more amino acid residues covalently linked by peptide bonds between the alpha-amino and carboxy groups of adjacent residues. In some embodiments, the polypeptide comprises a modified amino acid. In some embodiments, the polypeptide refers to a natural peptide, a recombinant peptide, or a combination thereof. In some embodiments, the polypeptide refers to short chains of amino acids. In some embodiments, the polypeptide refers to long chains of amino acids. In some embodiments, the polypeptide refers to a biologically active fragment, a substantially homologous polypeptide, an oligopeptide, a variant of a polypeptide, a modified polypeptide, a derivative, an analog, or a fusion protein. A person having ordinary skill in the biomedical art recognizes that individual substitutions, deletions, or additions to a peptide, polypeptide, or protein sequence which alters a single amino acid or a small percentage of amino acids in the encoded sequence are a conservatively modified variant where the alteration results in the substitution of amino acid with chemically similar amino acid and retains the desired activity of the polypeptide. Such conservatively modified variants also do not exclude polymorphic variants, interspecies homologs, and alleles consistent with the disclosure.

As used herein, the term “resistant” refers to a disease, e.g., cancer, that does not respond to a treatment. In some embodiments, the disease can be resistant to a treatment before or at the beginning of the treatment. In some embodiments, the cancer initially responds to the treatment but then becomes resistant during a treatment.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or geriatric subject. The human subject may be of either sex. In some embodiments, the subject may have a condition or disease or be at risk of developing a condition or disease.

As used herein, the terms “treat”, “treatment” and “treating” refer to the reversal, alleviation, amelioration, inhibition, reduction, slowing or halting of the progression, severity and/or duration of a disease, disorder, or medical condition, or the amelioration of one or more symptoms of a disease, disorder, or medical condition. In some embodiments, the disease, disorder, or medical condition is a proliferative disorder, such as growth of a tumor. In some embodiments, the terms “treat”, “treatment” and “treating” refer to the amelioration of at least one measurable physical parameter of the disease, disorder, or medical condition not necessarily discernible by the patient. In some embodiments, the terms “treat”, “treatment” and “treating” refer to the inhibition of the progression of disease, disorder, or medical condition, either physically by, e.g., stabilization of a discernible symptom, physiologically by, e.g., stabilization of a physical parameter, or both. In some embodiments, the terms “treat”, “treatment” and “treating” refer to the reduction or stabilization of tumor size or cancerous cell count. Treatment is effective, for example, if one or more symptoms or clinical markers are reduced or if the progression of a condition is reduced or halted. Treatment includes not just the improvement of symptoms or markers but also a cessation or at least slowing of progress or worsening of symptoms expected absent treatment.

As used herein, the term “heavy chain variable domain” (“VH”) refers to the variable region of the antibody heavy chain.

As used herein, the term “light chain variable domain” (“VL”) refers to the variable region of the antibody light chain.

This invention is not limited to the particular methodology, protocols, reagents, etc., described herein and as such can vary.

The disclosure described herein does not concern a process for cloning humans, processes for modifying the germ line genetic identity of humans, uses of human embryos for industrial or commercial purposes, or processes for modifying the genetic identity of animals likely to cause them suffering with no substantial medical benefit to man or animal, and animals resulting from such processes.

### DDR Inhibitors

### Ceralasertib

In some embodiments, the DDR inhibitor is a compound represented by Formula (I):

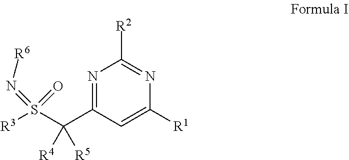

wherein:
R¹ is selected from morpholin-4-yl and 3-methylmorpholin-4-yl;
R² is

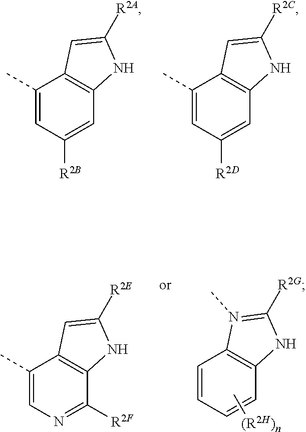

n is 0 or 1;
R^{2A}, R^{2C}, R^{2E} and R^{2F} each independently are hydrogen or methyl;
R^{2B} and R^{2D} each independently are hydrogen or methyl;
R^{2G} is selected from —NHR⁷ and —NHCOR⁸;
R^{2H} is fluoro;
R³ is methyl;
R⁴ and R⁵ are each independently hydrogen or methyl, or R⁴ and R⁵ together with the atom to which they are attached form Ring A;
Ring A is a C₃₋₆ cycloalkyl or a saturated 4-6 membered heterocyclic ring containing one heteroatom selected from O and N;
R⁶ is hydrogen;
R⁷ is hydrogen or methyl;
R⁸ is methyl, or a pharmaceutically acceptable salt thereof.

In some embodiments, the DDR inhibitor is a compound according to Formula I wherein R⁴ and R⁵ together with the atom to which they are attached form Ring A, and Ring A is a C₃₋₆cycloalkyl or a saturated 4-6 heterocyclic ring containing one heteroatom selected from O and N. In some embodiments Ring A is a cyclopropyl, tetrahydropyranyl or piperidinyl ring.

In some embodiments, in Formula I, R^{2A} is hydrogen; R^{2B} is hydrogen; R^{2C} is hydrogen; R^{2D} is hydrogen; R^{2E} is hydrogen; and R^{2F} is hydrogen.

In some embodiments, the DDR inhibitor is the compound in Formula I wherein R¹ is 3-methylmorpholin-4-yl, or a pharmaceutically acceptable salt thereof.

In some embodiments, the DDR inhibitor comprises ceralasertib. Ceralasertib has a chemical structure according to Formula II:

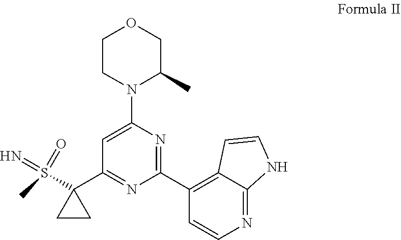 [SMILES: C[C@@H]1COCCN1c1cc(C2([S@](C)(=N)=O)CC2)nc(-c2ccnc3[nH]ccc23)n1]

In some embodiments, the ceralasertib is administered to the subject at a daily dose of between 10 mg and 100 mg, e.g., between 20 mg and 60 mg, e.g., between 30 mg and 50 mg.

### Camonsertib

In some embodiments, the DDR inhibitor comprises a compound of Formula III:

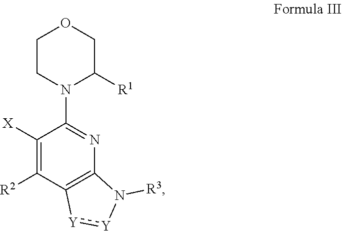

or a pharmaceutically acceptable salt thereof,
wherein [symbol]
is a double bond, and each Y is independently N or CR⁴; or [symbol]
is a single bond, and each Y is independently NR^{Y}, carbonyl, or C(R^{Y})₂; wherein each R^{Y} is independently H or optionally substituted C₁₋₆ alkyl;
R¹ is optionally substituted C₁₋₆ alkyl or H;
R₂ is

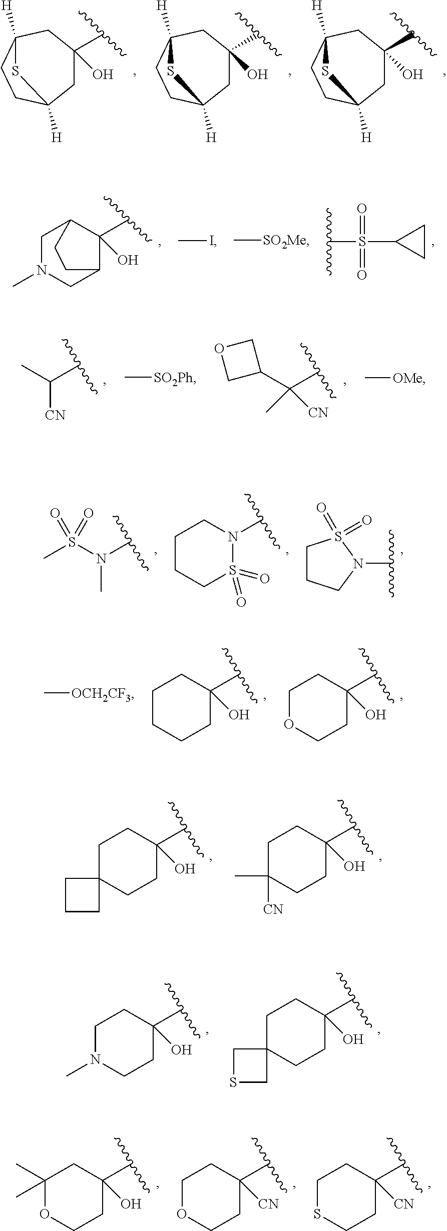

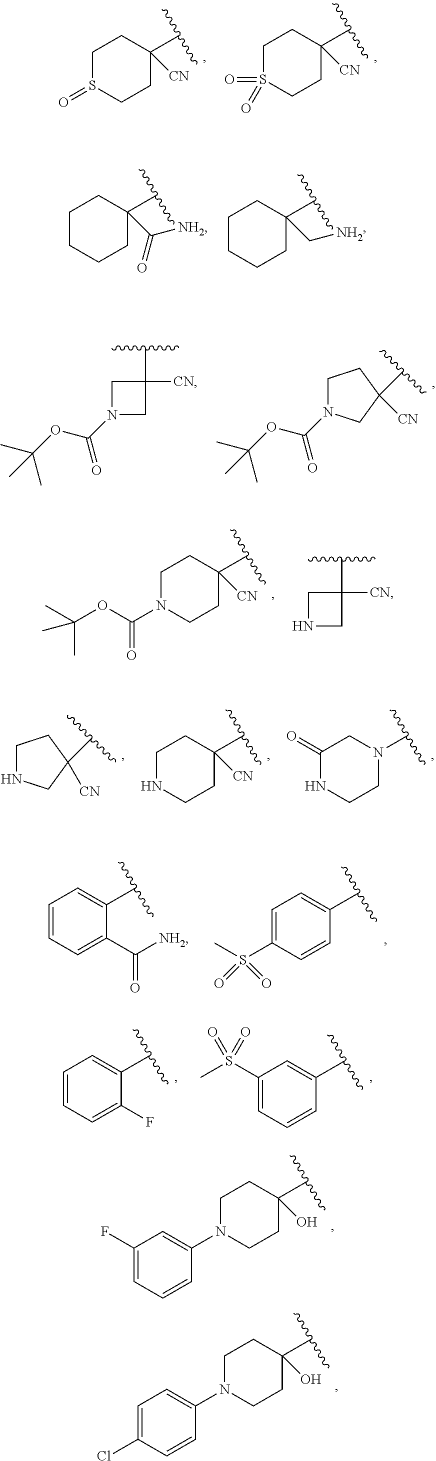

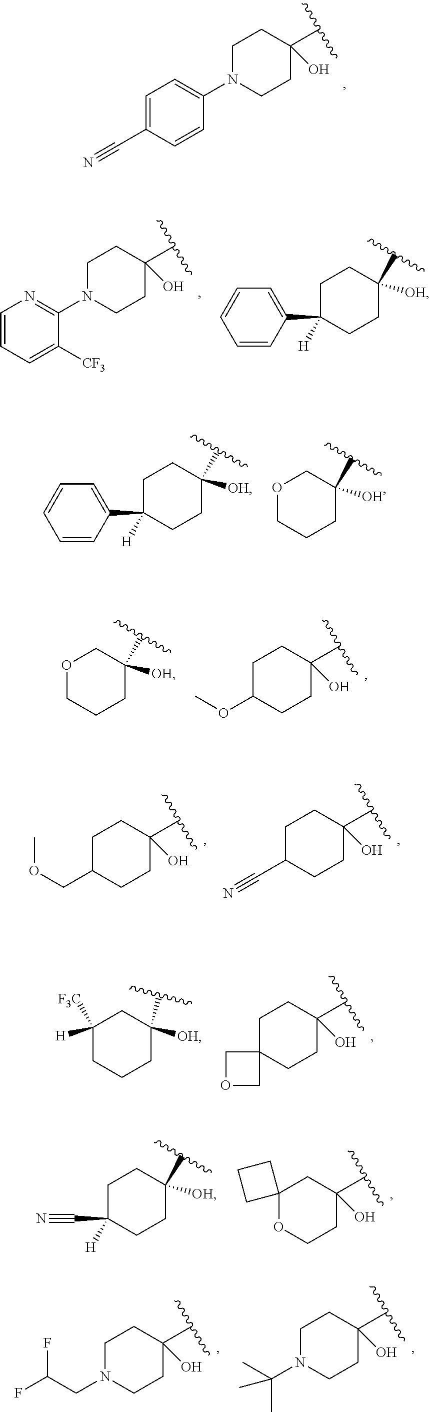

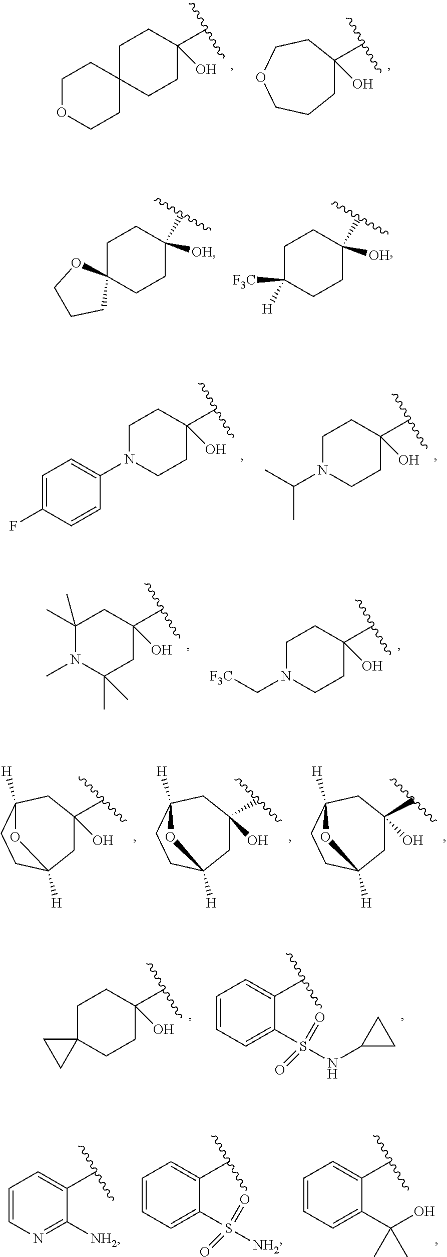

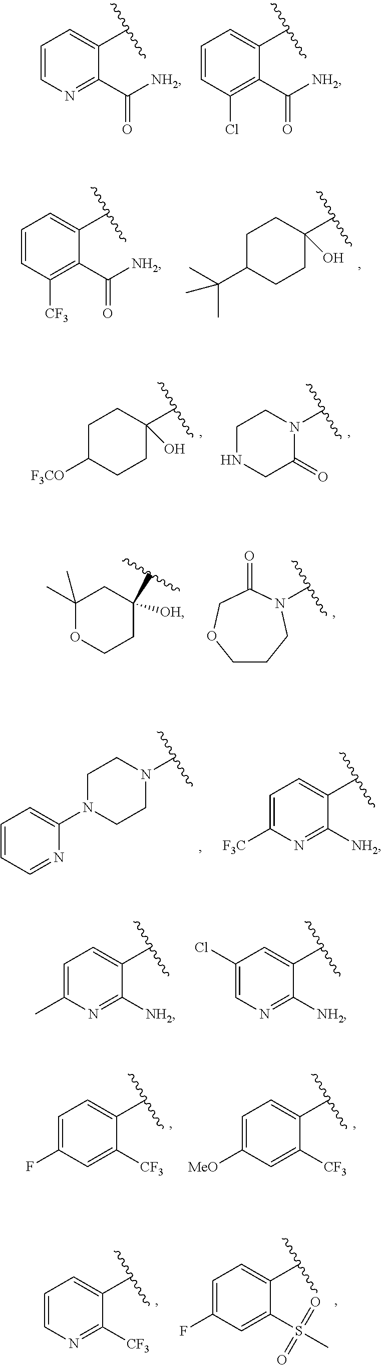

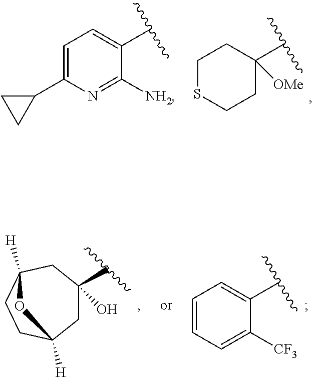

or R² is optionally substituted C₂, heterocyclyl, optionally substituted C₁₋₃ alkyl, optionally substituted C₃₋₈ cycloalkyl, optionally substituted C₂₋₉, heterocyclyl C₁₋₃ alkyl, optionally substituted C₆₋₁₀ aryl, optionally substituted C₁₋₉, heteroaryl, optionally substituted C₁₋₉, heteroaryl C₁₋₆ alkyl, halogen, N(R⁵)₂, OR⁵, CON(R⁶)₂, —SO₂N(R⁶)₂, —SO₂R^{5A}, or QR^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl; and
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or SO₂R^{5A}; or both R⁵, together with the atom to which they are attached, combine to form an optionally substituted C₂₋₉heterocyclyl;
each R^{5A} is independently optionally substituted C₁₋₆ alkyl, optionally substituted C₃₋₈ cycloalkyl, or optionally substituted C₃₋₁₀ aryl;
R^{5B} is hydroxyl, optionally substituted C₁₋₈ alkyl, optionally substituted C₆₋₁₀ aryl, optionally substituted C₁₋₉ heteroaryl, N (R⁵)₂, CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₈ alkoxyalkyl, optionally substituted C₆₋₁₀ aryl C₁₋₈ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene;
X is hydrogen or halogen.

In some embodiments, the DDR inhibitor comprises camonsertib. Camonsertib has a chemical structure according to Formula IV:

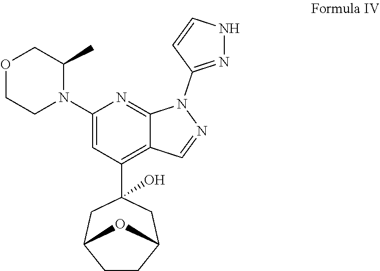 [SMILES: C[C@@H]1COCCN1c1cc([C@]2(O)C[C@H]3CC[C@@H](C2)O3)c2cnn(-c3cc[nH]n3)c2n1]

In some embodiments, the camonsertib is administered to the subject daily, e.g., at a daily dose of between 50 mg and 300 mg, e.g., between 100 mg and 200 mg, e.g. between 150 mg and 170 mg, optionally wherein the DDR inhibitor is administered daily for 3 days.

### Elimusertib

In some embodiments, the DDR inhibitor comprises elimusertib. Elimusertib has a chemical structure according to Formula V:

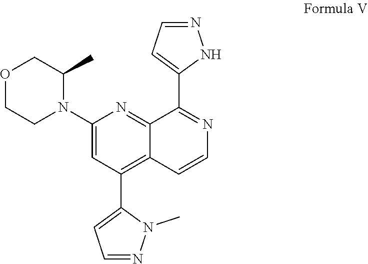 [SMILES: C[C@@H]1COCCN1c1cc(-c2ccnn2C)c2ccnc(-c3ccn[nH]3)c2n1]

In some embodiments, the elimusertib is administered to the subject daily, e.g., at a daily dose of between 20 mg and 200 mg, e.g. between 50 mg and 120 mg, e.g. between 70 mg and 90 mg, optionally wherein the DDR inhibitor is administered daily for 3 days.

### Berzosertib

In some embodiments, the DDR inhibitor is a compound of Formula VI:

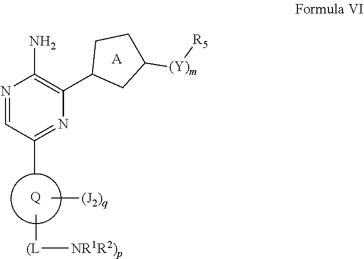

or a pharmaceutically acceptable salt thereof; wherein
Y is a C₁-C₁₀aliphatic chain wherein up to three methylene units of the aliphatic chain are optionally replaced with O, NR⁰, S, C(O) or S(O)₂;
Ring A is a 5 membered heteroaryl ring selected from

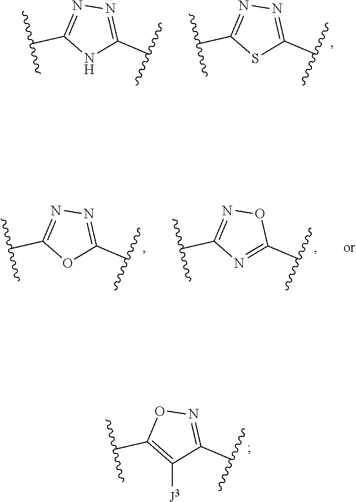

J₃ is H or C₁-C₄alkyl, wherein 1 methylene unit of the alkyl group can optionally be replaced with O, NH, N(C₁-C₄alkyl), or S and optionally substituted with 1-3 halo;
Q is a 5-6 membered monocyclic aromatic ring containing 0-4 heteroatoms independently selected from nitrogen, oxygen, and sulfur; or an 8-10 membered bicyclic aromatic ring containing 0-6 heteroatoms independently selected from nitrogen, oxygen, and sulfur;
R⁵ is H; a 3-7 membered monocyclic fully saturated, partially unsaturated, or aromatic ring containing 0-4 heteroatoms independently selected from nitrogen, oxygen, and sulfur; an 8-10 membered bicyclic fully saturated, partially unsaturated, or aromatic ring containing 0-6 heteroatoms independently selected from nitrogen, oxygen, and sulfur; wherein R⁵ is optionally substituted with 1-5 J⁵ groups;
L is a C₁-C₄alkyl chain wherein up to two methylene units of the alkyl chain are optionally replaced with O, NR⁶, S, —C(O)—, —SO—, or SO₂—;
R⁰ is H C₁-C₆alkyl wherein one methylene unit of the alkyl chain can be optionally replaced with O, NH, N(C₁-C₄alkyl), or S;
R¹ is H or C₁-C₆alkyl;
R² is H, C₁-C₆alkyl, —(C₂-C₆alkyl)-Z or a 4-8 membered cyclic ring containing 0-2 nitrogen atoms; wherein said ring is bonded via a carbon atom and is optionally substituted with one occurrence of J^{Z};
or R¹ and R², taken together with the atom to which they are bound, form a 4-8 membered heterocyclic ring containing 1-2 heteroatoms selected from oxygen, nitrogen, and sulfur;
wherein said heterocyclic ring is optionally substituted with one occurrence of J^{Z1};
J^{Z1} is halo, CN, C₁-C₈aliphatic, —(X)ₜ—CN, or —(X)ₜ—Z, wherein said up to two methylene units of said C₁-C₈aliphatic can be optionally replaced with O, NR, S, P(O), C(O), S(O), or S (O)₂, wherein said C₁-C₈aliphatic is optionally substituted with halo, CN, or NO₂;
X is C₁-C₄alkyl;
each t, r and m is independently 0 or 1;
Z is —NR³R⁴;
R³ is H or C₁-C₂alkyl;
R⁴ is H or C₁-C₆alkyl;
or R³ and R⁴, taken together with the atom to which they are bound, form a 4-8 membered heterocyclic ring containing 1-2 heteroatoms selected from oxygen, nitrogen, and sulfur; wherein said ring is optionally substituted with one occurrence of J^{Z};
R⁶ is H, or C₁-C₆alkyl;
J^{Z} is independently NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO(C₁-C₄aliphatic), CO₂(C₁-C₄aliphatic), O(haloC₁-C₄aliphatic), or haloC₁-C₄aliphatic;
J⁵ is halo, oxo, CN, NO₂, X¹—R, or —(X¹)ₚ-Q₄;
X¹is C₁-C₁₀aliphatic; wherein 1-3 methylene units of said C₁-C₁₀aliphatic are optionally replaced with —NR′—, —O—, —S—, C(═NR′), C(O), S(O)₂, or S(O), wherein X¹ is optionally and independently substituted with 1-4 occurrences of NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO₂(C₁-C₄aliphatic), C(O)NH₂, C(O)NH(C₁-C₄aliphatic), C(O)N(C₁-C₄aliphatic)₂, SO(C₁-C₄aliphatic), SO₂(C₁-C₄aliphatic), SO₂NH(C₁-C₄aliphatic), NHC(O)(C₁-C₄aliphatic), N(C₁-C₄aliphatic)C(O)(C₁-C₄aliphatic), wherein said C₁-C₄aliphatic is optionally substituted with 1-3 occurrences of halo;
Q⁴ is a 3-8 membered saturated or unsaturated monocyclic ring having 0-4 heteroatoms independently selected from nitrogen, oxygen, and sulfur, or an 8-10 membered saturated or unsaturated bicyclic ring having 0-6 heteroatoms independently selected from nitrogen, oxygen, and sulfur; each Q⁴ is optionally substituted with 1-5 J^{Q4};
J^{Q4} is halo, CN, or C₁-C₄alkyl wherein up to 2 methylene units are optionally replaced with O, NR*, S, C(O), S(O), or S(O)₂;
R is H or C₁-C₄alkyl wherein said C₁-C₄alkyl is optionally substituted with 1-4 halo;
J² is halo; CN; a 5-6 membered aromatic or nonaromatic monocyclic ring having 0-3 heteroatoms selected from oxygen, nitrogen, and sulfur; or a C₁-C₁₀aliphatic group wherein up to 2 methylene units are optionally replaced with O, NR″, C(O), S, S(O), or S(O)₂; wherein said C₁-C₁₀aliphatic group is optionally substituted with 1-3 halo or CN; and said monocyclic ring is optionally substituted with 1-3 occurrences of halo; CN; a C₃-C₆cycloalkyl; a 3-7 membered heterocyclyl containing 0-2 heteroatoms selected from oxygen, nitrogen, and sulfur; or a C₁-C₄alkyl wherein up to one methylene unit of the alkyl chain is optionally replaced with O, NR″, or S; and wherein said C₁-C₄alkyl is optionally substituted with 1-3 halo;
q is 0, 1, or 2;
p is 0 or 1;
R′, R″, and R* are each independently H, C₁-C₄alkyl, or is absent; wherein said C₁-C₄alkyl is optionally substituted with 1-4 halo.

In some embodiments, the DDR inhibitor comprises berzosertib. Berzosertib has a chemical structure according to Formula VII:

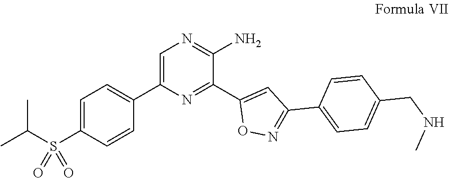 [SMILES: CNCc1ccc(-c2cc(-c3nc(-c4ccc(S(=O)(=O)C(C)C)cc4)cnc3N)on2)cc1]

In some embodiments, the berzosertib is administered to the subject at a daily dose of between 100 mg/m² and 400 mg/m², e.g. between 150 mg/m² and 250 mg/m², e.g. between 200 mg/m² and 220 mg/m² per day.

### GDF15 Inhibitors

Growth differentiation factor 15 (GDF15) is a TGF-beta ligand. GDF15 is thought to have a role in cancer, especially with respect to chances of metastasis, overall survival, and weight loss.

### Ponsegromab

In some embodiments, the GDF15 inhibitor comprises an antibody molecule having one or more of (e.g., 1, 2, 3, 4, 5, or all of) a light chain complementary determining region 1 (LC CDR1), a light chain complementary determining region 2 (LC CDR2), a light chain complementary determining region 3 (LC CDR3), a heavy chain complementary determining region 1 (HC CDR1), a heavy chain complementary determining region 2 (HC CDR2), and a heavy chain complementary determining region 3 (HC CDR3) having amino acid sequences according to Table 1.

TABLE 1
CDRs of ponsegromab

| | |
|---|---|
| HC CDR1 | GYTFSSYNID (SEQ ID NO: 1) |
| HC CDR2 | GINPIFGTAFYNQKFQG (SEQ ID NO: 2) |
| HC CDR3 | EAITTVGAMDH (SEQ ID NO: 3) |
| LC CDR1 | RTSQSVHNYLA (SEQ ID NO: 4) |
| LC CDR2 | DASTRAD (SEQ ID NO: 5) |
| LC CDR3 | QQFWSWPWT (SEQ ID NO: 6) |

In some embodiments, the GDF15 inhibitor comprises an antibody molecule having a heavy chain variable region (VH) comprising a VH amino acid sequence according to Table 2, or a sequence having at least 70%, 75%, 80%, 85%, 90%, 95%, 97%, 98%, or 99% identity thereto. In some embodiments, the GDF15 inhibitor comprises an antibody molecule having a light chain variable region (VL) comprising a VL amino acid sequence according to Table 2, or a sequence having at least 70%, 75%, 80%, 85%, 90%, 95%, 97%, 98%, or 99% identity thereto. In some embodiments, the GDF15 inhibitor comprises an antibody molecule having a heavy chain (HC) comprising a HC amino acid sequence according to Table 2, or a sequence having at least 70%, 75%, 80%, 85%, 90%, 95%, 97%, 98%, or 99% identity thereto. In some embodiments, the GDF15 inhibitor comprises an antibody molecule having a light chain (LC) comprising a LC amino acid sequence according to Table 2, or a sequence having at least 70%, 75%, 80%, 85%, 90%, 95%, 97%, 98%, or 99% identity thereto.

TABLE 2
VH, VL, HC, and LC of ponsegromab

| | |
|---|---|
| VH | QVQLVQSGAEVKKPGSSVKVSCKASGYTFS |
| | SYNIDWVRQAPGQGLEWMGGINPIFGTAFY |
| | NQKFQGRVTITADESTSTAYMELSSLRSED |
| | TAVYYCAREAITTVGAMDHWGQGTLVTVSS |
| | (SEQ ID NO: 7) |
| VL | EIVLTQSPATLSLSPGERATLSCRTSQSVH |
| | NYLAWYQQKPGQAPRLLIYDASTRADGIPA |
| | RFSGSGSGTDFTLTISSLEPEDFAVYYCQQ |
| | FWSWPWTFGQGTKVEIK |
| | (SEQ ID NO: 8) |
| HC | QVQLVQSGAEVKKPGSSVKVSCKASGYTFS |
| | SYNIDWVRQAPGQGLEWMGGINPIFGTAFY |
| | NQKFQGRVTITADESTSTAYMELSSLRSED |
| | TAVYYCAREAITTVGAMDHWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSREE |
| | MTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPG |
| | (SEQ ID NO: 9) |
| LC | EIVLTQSPATLSLSPGERATLSCRTSQSVH |
| | NYLAWYQQKPGQAPRLLIYDASTRADGIPA |
| | RFSGSGSGTDFTLTISSLEPEDFAVYYCQQ |
| | FWSWPWTFGQGTKVEIKRTVAAPSVFIFPP |
| | SDEQLKSGTASVVCLLNNFYPREAKVQWKV |
| | DNALQSGNSQESVTEQDSKDSTYSLSSTLT |
| | LSKADYEKHKVYACEVTHQGLSSPVTKSFN |
| | RGEC |
| | (SEQ ID NO: 10) |

In some embodiments, the GDF15 inhibitor comprises ponsegromab. In some embodiments, the GDF15 inhibitor (e.g., ponsegromab) is administered intravenously (IV) or subcutaneously (SC).

In some embodiments, the GDF15 inhibitor (e.g., ponsegromab) is administered in an initial dose of about 0.025 mg/kg to about 20 mg/kg. The initial dose may be followed by one or more subsequent doses. In some embodiments, one or more subsequent doses may be administered at least any of weekly, every other week, every three weeks, every four weeks, every five weeks, every six weeks, every seven weeks, every eight weeks, every nine weeks, every ten weeks, every eleven weeks, or every twelve weeks.

In some embodiments, the GDF15 inhibitor (e.g., ponsegromab) is administered as a fixed dose of about 0.25 mg to about 2000 mg. In some embodiments, the GDF15 inhibitor (e.g., ponsegromab) is administered weekly, every other week, every three weeks, every four weeks, every five weeks, every six weeks, every seven weeks, every eight weeks, every nine weeks, every ten weeks, every eleven weeks, or every twelve weeks.

In some embodiments, the GDF15 inhibitor (e.g., ponsegromab) is administered as a fixed dose of about 0.1 to about 60 mg every week. In some embodiments, the GDF15 inhibitor (e.g., ponsegromab) is administered as a fixed dose of about 2 mg, about 5 mg, about 7 mg, about 10 mg, about 12 mg, about 15 mg, about 25 mg, about 40 mg, and about 50 mg weekly.

In some embodiments, the GDF15 inhibitor (e.g., ponsegromab) is administered as a fixed dose of about 0.1 to about 130 mg every other week. In some embodiments, the GDF15 inhibitor (e.g., ponsegromab) is administered as a fixed dose of about 5 mg, about 10 mg, about 12 mg, about 15 mg, about 20 mg, about 25 mg, about 30 mg, about 35 mg, about 40 mg, about 50 mg, about 60 mg, about 70 mg, about 75 mg, about 80 mg, about 90 mg, about 100 mg, about 110 mg, about 120 mg and about 125 mg bi-weekly.

In some embodiments, the GDF15 inhibitor (e.g., ponsegromab) is administered as a fixed dose of about 0.1 to about 400 mg every 21 days (±2 days).

In some embodiments, the GDF15 inhibitor (e.g., ponsegromab) is administered as a fixed dose in about 15 mg, about 25 mg, about 30 mg, about 40 mg, about 50 about 60 mg, about 75 mg, about 100 mg, about 115 mg, about 125 mg, about 150 mg, about 175 mg, about 200 mg, about 225 mg, about 250 mg, about 275 mg, about 300 mg, about 325 mg, about 350 mg, about 375 mg and about 385 mg administered every 21 days (±2 days).

In some embodiments, the GDF15 inhibitor (e.g., ponsegromab) is administered as a fixed dose of about 0.1 to about 400 mg every 28 days (+2 days). In some embodiments, the GDF15 inhibitor (e.g., ponsegromab) is administered as a fixed dose in about 15 mg, about 25 mg, about 30 mg, about 40 mg, about 50 mg, about 60 mg, about 75 mg, about 100 mg, about 115 mg, about 125 mg, about 150 mg, about 175 mg, about 200 mg, about 225 mg, about 250 mg, about 275 mg, about 300 mg, about 325 mg, about 350 mg, about 375 mg and about 385 mg administered every 28 days (+2 days).

### AV-380

In some embodiments, the GDF15 inhibitor comprises an antibody molecule having one or more of (e.g., 1, 2, 3, 4, 5, or all of) a light chain complementary determining region 1 (LC CDR1), a light chain complementary determining region 2 (LC CDR2), a light chain complementary determining region 3 (LC CDR3), a heavy chain complementary determining region 1 (HC CDR1), a heavy chain complementary determining region 2 (HC CDR2), and a heavy chain complementary determining region 3 (HC CDR3) having amino acid sequences according to Tables 3 and 4.

TABLE 3
Heavy chain CDR sequences (Kabat, Chothia, and IMGT definitions)

| | | |
|---|---|---|
| CDR1 | CDR2 | CDR3 |

Kabat

| | | | |
|---|---|---|---|
| 01G06 | DYNMD (SEQ | QINPNNGGIFFNQKFKG | EAITTVGAMDY |
| | ID NO: 11) | (SEQ ID NO: 12) | (SEQ ID NO: 13) |
| 03G05 | SYWIH (SEQ | DINPSNGRSKYNEKFKN | EVLDGAMDY |
| | ID NO: 14) | (SEQ ID NO: 15) | (SEQ ID NO: 16) |
| 04F08 | TYGMGVT | HIYWDDDKRYNPSLKS | TGYSNLFAY |
| | (SEQ ID NO: | (SEQ ID NO: 18) | (SEQ ID NO: |
| | 17) | | 19) |
| 06C11 | TYGMGVS | HIYWDDDKRYNPSLKS | RGYDDYWGY |
| | (SEQ ID NO: | (SEQ ID NO: 18) | (SEQ ID NO: 21) |
| | 20) | | |
| 08G01 | DYNMD (SEQ | EINPNNGGTFYNQKFKG | EAITTVGAMDY |
| | ID NO: 11) | (SEQ ID NO: 22) | (SEQ ID NO: 13) |
| 14F11 | TYGMGVG | DIWWDDDKYYNPSLKS | RGHYSAMDY |
| | (SEQ ID NO: | (SEQ ID NO: 24) | (SEQ ID NO: 25) |
| | 23) | | |
| 17B11 | TSGMGVS | HNDWDDDKRYKSSLKS | RVGGLEGYFDY |
| | (SEQ ID NO: | (SEQ ID NO: 27) | (SEQ ID NO: 28) |
| | 26) | | |
| Sh01G06 IGHVI-18 | DYNMD (SEQ | QINPNNGGIFFNQKFQG | EAITTVGAMDY |
| M69L K64Q G44S | ID NO: 11) | (SEQ ID NO: 29) | (SEQ ID NO: 13) |
| Hu01G06 IGHV1-18 | DYNMD (SEQ | QINPYNHLIFFNQKFQG | EAITTVGAMDY |
| F1 | ID NO: 11) | (SEQ ID NO: 30) | (SEQ ID NO: 13) |
| Hu01G06 IGHV1-18 | DYNMD (SEQ | QINPNNGLIFFNQKFQG | EAITTVGAMDY |
| F2 | ID NO: 11) | (SEQ ID NO: 31) | (SEQ ID NO: 13) |
| Hu01G06 IGHVI69 | DYNMD (SEQ | QINPNNGLIFFNQKFKG | EAITTVGAMDY |
| FI | ID NO: 11) | (SEQ ID NO: 32) | (SEQ ID NO: 13) |
| Hu01G06 IGHVI69 | DYNMD (SEQ | QINPYNHLIFFNQKFKG | EAITTVGAMDY |
| F2 | ID NO: 11) | (SEQ ID NO: 33) | (SEQ ID NO: 13) |
| Table 9, sequence 8 | TYGMGVS | HIYWDDDKRYNPSLKT | RGYDDYWGY |
| from | (SEQ ID NO: | (SEQ ID NO: 34) | (SEQ ID NO: 21) |
| US20170137506 | 20) | | |

Chothia

| | | | |
|---|---|---|---|
| 01G06 | GYTFTDY | NPNNGG (SEQ ID NO: 36) | EAITTVGAMDY |
| | (SEQ ID NO: | | (SEQ ID NO: 13) |
| | 35) | | |
| 03G05 | GYTFTSY | NPSNGR (SEQ ID NO: 38) | EVLDGAMDY |
| | (SEQ ID NO: | | (SEQ ID NO: 16) |
| | 37) | | |
| 04F08 | GFSLSTYGM | YWDDD (SEQ ID NO: 40) | TGYSNLFAY (SEQ |
| | (SEQ ID NO: | | ID NO: 19) |
| | 39) | | |
| 06C11 | GFSLNTYGM | YWDDD (SEQ ID NO: 40) | RGYDDYWGY |
| | (SEQ ID NO: | | (SEQ ID NO: 21) |
| | 41) | | |
| 08G01 | GYTFTDY | NPNNGG (SEQ ID NO: 36) | EAITTVGAMDY |
| | (SEQ ID NO: | | (SEQ ID NO: 13) |
| | 35) | | |
| 14F11 | GFSLSTYGM | WWDDD (SEQ ID NO: 42) | RGHYSAMDY |
| | (SEQ ID NO: | | (SEQ ID NO: 25) |
| | 39) | | |
| 17B11 | GFSLSTSGM | DWDDD (SEQ ID NO: 44) | RVGGLEGYFDY |
| | (SEQ ID NO: | | (SEQ ID NO: 28) |
| | 43) | | |
| Sh01G06 IGHVI69 | GYTFSDY | NPNNGG (SEQ ID NO: 36) | EAITTVGAMDY |
| T30S 169L | (SEQ ID NO: | | (SEQ ID NO: 13) |
| | 45) | | |
| Hu01G06 IGHV1- | GYTFTDY | NPYNHL (SEQ ID NO: 46) | EAITTVGAMDY |
| 18 F1 | (SEQ ID NO: | | (SEQ ID NO: 13) |
| | 35) | | |
| Hu01G06 IGHV1- | GYTFTDY | NPNNGL (SEQ ID NO: 47) | EAITTVGAMDY |
| 18 F2 | (SEQ ID NO: | | (SEQ ID NO: 13) |
| | 35) | | |
| Hu01G06 IGHVI69 | GYTFSDY | NPNNGL (SEQ ID NO: 47) | EAITTVGAMDY |
| FI | (SEQ ID NO: | | (SEQ ID NO: 13) |
| | 45) | | |
| Hu01G06 IGHVI69 | GYTFSDY | NPYNHL (SEQ ID NO: 46) | EAITTVGAMDY |
| F2 | (SEQ ID NO: | | (SEQ ID NO: 13) |
| | 45) | | |

IMGT

| | | | |
|---|---|---|---|
| 01G06 | GYTFTDYN | INPNNGGI (SEQ ID NO: | AREAITTVGAMDY |
| | (SEQ ID NO: | | |
| | 48) | 49) | (SEQ ID NO: 50) |
| 03G05 | GYTFTSYW | INPSNGRS (SEQ ID NO: | AREVLDGAMDY |
| | (SEQ ID NO: | 52) | (SEQ ID NO: 53) |
| | 51) | | |
| 04F08 | GFSLSTYGMG | IYWDDDK (SEQ ID NO: | AQTGYSNLFAY |
| | (SEQ ID NO: | 55) | (SEQ ID NO: 56) |
| | 54) | | |
| 06C11 | GFSLNTYGMG | IYWDDDK (SEQ ID NO: | AQRGYDDYWGY |
| | (SEQ ID NO: | 55) | (SEQ ID NO: 58) |
| | 57) | | |
| 08G01 | GYTFTDYN | INPNNGGT (SEQ ID NO: | AREAITTVGAMDY |
| | (SEQ ID NO: | 59) | (SEQ ID NO: 50) |
| | 48) | | |
| 14F11 | GFSLSTYGMG | IWWDDDK (SEQ ID NO: | ARRGHYSAMDY |
| | (SEQ ID NO: | 60) | (SEQ ID NO: 61) |
| | 54) | | |
| 17B11 | GFSLSTSGMG | NDWDDDK (SEQ ID NO: | ARRVGGLEGYFDY |
| | (SEQ ID NO: | 63) | (SEQ ID NO: 64) |
| | 62) | | |
| Sh01G06 IGHVI69 | GYTFSDYN | INPNNGGI (SEQ ID NO: | AREAITTVGAMDY |
| T30S 169L | (SEQ ID NO: | 49) | (SEQ ID NO: 50) |
| | 65) | | |
| Hu01G06 IGHV1- | GYTFTDYN | INPYNHLI (SEQ ID NO: | AREAITTVGAMDY |
| 18 F1 | (SEQ ID NO: | 66) | (SEQ ID NO: 50) |
| | 48) | | |
| Hu01G06 IGHV1- | GYTFTDYN | INPNNGLI (SEQ ID NO: | AREAITTVGAMDY |
| 18 F2 | (SEQ ID NO: | 67) | (SEQ ID NO: 50) |
| | 48) | | |
| Hu01G06 IGHVI69 | GYTFSDYN | INPNNGLI (SEQ ID NO: | AREAITTVGAMDY |
| FI | (SEQ ID NO: | 67) | (SEQ ID NO: 50) |
| | 65) | | |
| Hu01G06 IGHVI69 | GYTFSDYN | INPYNHLI (SEQ ID NO: | AREAITTVGAMDY |
| F2 | (SEQ ID NO: | 66) | (SEQ ID NO: 50) |
| | 65) | | |

TABLE 4
Light chain CDR sequences (Kabat, Chothia, and IMGT definitions)

| | | |
|---|---|---|
| CDR1 | CDR2 | CDR3 |

Kabat/Chothia

| | | | |
|---|---|---|---|
| 01G06 | RTSENLHNYLA | DAKTLAD (SEQ | QHFWSSPYT |
| | (SEQ ID NO: 68) | ID NO: 69) | (SEQ ID NO: 70) |
| 03G05 | RASESVDNYGISFMN | AASNQGS (SEQ | QQSKEVPWT |
| | (SEQ ID NO: 71) | ID NO: 72) | (SEQ ID NO: 73) |
| 04F08 | KASQNVGTNVA | SASYRYS (SEQ ID | QQYNSYPYT |
| | (SEQ ID NO: 74) | NO: 75) | (SEQ ID NO: 76) |
| 06C11 | KASQNVGTNVA | SASYRYS (SEQ ID | QQYNNYPLT |
| | (SEQ ID NO: 74) | NO: 75) | (SEQ ID NO: 77) |
| 08G01 | RASGNIHNYLA | NAKTLAD (SEQ | QHFWSSPYT |
| | (SEQ ID NO: 78) | ID NO: 79) | (SEQ ID NO: 70) |
| 14F11 | KASQNVGTNVA | SPSYRYS (SEQ ID | QQYNSYPHT |
| | (SEQ ID NO: 74) | NO: 80) | (SEQ ID NO: 81) |
| 17B11 | RASQSVSTSRFSYMH | YASNLES (SEQ ID | QHSWEIPYT |
| | (SEQ ID NO: 82) | NO: 83) | (SEQ ID NO: 84) |
| Table 2, | RTSENLHNYLA (SEQ | DAKTLAD (SEQ | QHFWSDPYT |
| sequence 2 from | ID NO: 68) | ID NO: 69) | (SEQ ID NO: 85) |
| US20170137506 | | | |

IMGT

| | | | |
|---|---|---|---|
| 01G06 | ENLHNY (SEQ ID | DAK | QHFWSSPYT |
| | NO: 86) | | (SEQ ID NO: 70) |
| 03G05 | ESVDNYGISF (SEQ | AAS | QQSKEVPWT |
| | ID NO: 87) | | (SEQ ID NO: 73) |
| 04F08 | QNVGTN (SEQ ID | SAS | QQYNSYPYT |
| | NO: 88) | | (SEQ ID NO: 76) |
| 06C11 | QNVGTN (SEQ ID | SAS | QQYNNYPLT |
| | NO: 88) | | (SEQ ID NO: 77) |
| 08G01 | GNIHNY (SEQ ID NO: | NAK | QHFWSSPYT |
| | 89) | | (SEQ ID NO: 70) |
| 14F11 | QNVGTN (SEQ ID | SPS | QQYNSYPHT |
| | NO: 88) | | (SEQ ID NO: 81) |
| 17B11 | QSVSTSRFSY (SEQ | YAS | QHSWEIPYT |
| | ID NO: 90) | | (SEQ ID NO: 84) |

In some embodiments, the GDF15 inhibitor comprises an antibody molecule having a heavy chain variable region (VH) comprising a VH amino acid sequence according to Table 5, or a sequence having at least 70%, 75%, 80%, 85%, 90%, 95%, 97%, 98%, or 99% identity thereto. In some embodiments, the GDF15 inhibitor comprises an antibody molecule having a light chain variable region (VL) comprising a VL amino acid sequence according to Table 5, or a sequence having at least 70%, 75%, 80%, 85%, 90%, 95%, 97%, 98%, or 99% identity thereto. In some embodiments, the GDF15 inhibitor comprises an antibody molecule having a heavy chain (HC) comprising a HC amino acid sequence according to Table 5, or a sequence having at least 70%, 75%, 80%, 85%, 90%, 95%, 97%, 98%, or 99% identity thereto. In some embodiments, the GDF15 inhibitor comprises an antibody molecule having a light chain (LC) comprising a LC amino acid sequence according to Table 5, or a sequence having at least 70%, 75%, 80%, 85%, 90%, 95%, 97%, 98%, or 99% identity thereto.

TABLE 5
VH, VL, HC, and LC of anti GDF 15 antibodies

| | | |
|---|---|---|
| 01G06 | VH | EVLLQQSGPELVKPGASVKIPCKASGYTFT |
| | | DYNMDWVKQSHGKSLEWIGQINPNNGGIFF |
| | | NQKFKGKATLTVDKSSNTAFMEVRSLTSED |
| | | TAVYYCAREAITTVGAMDYWGQGTSVTVSS |
| | | (SEQ ID NO: 91) |
| | VL | DIQMTQSPASLSASVGETVTITCRTSENLH |
| | | NYLAWYQQKQGKSPQLLVYDAKTLADGVPS |
| | | RFSGSGSGTQYSLKINSLQPEDFGSYYCQH |
| | | FWSSPYTFGGGTKLEIK |
| | | (SEQ ID NO: 92) |
| | HC | EVLLQQSGPELVKPGASVKIPCKASGYTFT |
| | | DYNMDWVKQSHGKSLEWIGQINPNNGGIFF |
| | | NQKFKGKATLTVDKSSNTAFMEVRSLTSED |
| | | TAVYYCAREAITTVGAMDYWGQGTSVTVSS |
| | | AKTTPPSVYPLAPGSAAQTNSMVTLGCLVK |
| | | GYFPEPVTVTWNSGSLSSGVHTFPAVLQSD |
| | | LYTLSSSVTVPSSTWPSETVTCNVAHPASS |
| | | TKVDKKIVPRDCGCKPCICTVPEVSSVFIF |
| | | PPKPKDVLTITLTPKVTCVVVDISKDDPEV |
| | | QFSWFVDDVEVHTAQTQPREEQFNSTFRSV |
| | | SELPIMHQDWLNGKEFKCRVNSAAFPAPIE |
| | | KTISKTKGRPKAPQVYTIPPPKEQMAKDKV |
| | | SLTCMITDFFPEDITVEWQWNGQPAENYKN |
| | | TQPIMDTDGSYFVYSKLNVQKSNWEAGNTF |
| | | TCSVLHEGLHNHHTEKSLSHSPG |
| | | K |
| | | (SEQ ID NO: 93) |
| | LC | DIQMTQSPASLSASVGETVTITCRTSENLH |
| | | NYLAWYQQKQGKSPQLLVYDAKTLADGVPS |
| | | RFSGSGSGTQYSLKINSLQPEDFGSYYCQH |
| | | FWSSPYTFGGGTKLEIKRADAAPTVSIFPP |
| | | SSEQLTSGGASVVCFLNNFYPKDINVKWKI |
| | | DGSERQNGVLNSWTDQDSKDSTYSMSSTLT |
| | | LTKDEYERHNSYTCEATHKTSTSPIVKSFN |
| | | RNEC |
| | | (SEQ ID NO: 94) |
| 03G05 | VH | QVQLQQPGAELVKPGASVKLSCKASGYTFT |
| | | SYWIHWVNQRPGQGLEWIGDINPSNGRSKY |
| | | NEKFKNKATMTADKSSNTAYMQLSSLTSED |
| | | SAVYYCAREVLDGAMDYWGQGTSVTVSS |
| | | (SEQ ID NO: 95) |
| | VL | DIVLTQSPASLAVSLGQRATISCRASESVD |
| | | NYGISFMNWFQQKPGQPPKLLIYAASNQGS |
| | | GVPARFSGSGSGTDFSLNIHPMEEDDTAMY |
| | | FCQQSKEVPWTFGGGSKLEIK |
| | | (SEQ ID NO: 96) |
| | HC | QVQLQQPGAELVKPGASVKLSCKASGYTFT |
| | | SYWIHWVNQRPGQGLEWIGDINPSNGRSKY |
| | | NEKFKNKATMTADKSSNTAYMQLSSLTSED |
| | | SAVYYCAREVLDGAMDYWGQGTSVTVSSAK |
| | | TTPPSVYPLAPGSAAQTNSMVTLGCLVKGY |
| | | FPEPVTVTWNSGSLSSGVHTFPAVLQSDLY |
| | | TLSSSVTVPSSTWPSETVTCNVAHPASSTK |
| | | VDKKIVPRDCGCKPCICTVPEVSSVFIFPP |
| | | KPKDVLTITLTPKVTCVVVDISKDDPEVQF |
| | | SWFVDDVEVHTAQTQPREEQFNSTFRSVSE |
| | | LPIMHQDWLNGKEFKCRVNSAAFPAPIEKT |
| | | ISKTKGRPKAPQVYTIPPPKEQMAKDKVSL |
| | | TCMITDFFPEDITVEWQWNGQPAENYKNTQ |
| | | PIMDTDGSYFVYSKLNVQKSNWEAGNTFTC |
| | | SVLHEGLHNHHTEKSLSHSPGK |
| | | (SEQ ID NO: 97) |
| | LC | DIVLTQSPASLAVSLGQRATISCRASESVD |
| | | NYGISFMNWFQQKPGQPPKLLIYAASNQGS |
| | | GVPARFSGSGSGTDFSLNIHPMEEDDTAMY |
| | | FCQQSKEVPWTFGGGSKLEIKRADAAPTVS |
| | | IFPPSSEQLTSGGASVVCFLNNFYPKDINV |
| | | KWKIDGSERQNGVLNSWTDQDSKDSTYSMS |
| | | STLTLTKDEYERHNSYTCEATHKTSTSPIV |
| | | KSFNRNEC |
| | | (SEQ ID NO: 98) |
| 04F08 | VH | QVTLKESGPGILQPSQTLSLTCSFSGFSLS |
| | | TYGMGVTWIRQPSGKGLEWLAHIYWDDDKR |
| | | YNPSLKSRLTISKDTSNNQVFLKITSVDTA |
| | | DTATYYCAQTGYSNLFAYWGQGTLVTVSA |
| | | (SEQ ID NO: 99) |
| | VL | DIVMTQSQKFMSTSVGDRVSVTCKASQNVG |
| | | TNVAWYQQKLGQSPKTLIYSASYRYSGVPD |
| | | RFTGSGSGTDFTLTISNVQSEDLAEYFCQQ |
| | | YNSYPYTFGGGTKLEIK |
| | | (SEQ ID NO: 100) |
| | HC | QVTLKESGPGILQPSQTLSLTCSFSGFSLS |
| | | TYGMGVTWIRQPSGKGLEWLAHIYWDDDKR |
| | | YNPSLKSRLTISKDTSNNQVFLKITSVDTA |
| | | DTATYYCAQTGYSNLFAYWGQGTLVTVSAA |
| | | KTTPPSVYPLAPGSAAQTNSMVTLGCLVKG |
| | | YFPEPVTVTWNSGSLSSGVHTFPAVLQSDL |
| | | YTLSSSVTVPSSTWPSETVTCNVAHPASST |
| | | KVDKKIVPRDCGCKPCICTVPEVSSVFIFP |
| | | PKPKDVLTITITPKVTCVVVDISKDDPEVQ |
| | | FSWFVDDVEVHTAQTQPREEQFNSTFRSVS |
| | | ELPIMHQDWLNGKEFKCRVNSAAFPAPIEK |
| | | TISKTKGRPKAPQVYTIPPPKEQMAKDKVS |
| | | LTCMITDFFPEDITVEWQWNGQPAENYKNT |
| | | QPIMDTDGSYFVYSKLNVQKSNWEAGNTFT |
| | | CSVLHEGLHNHHTEKSLSHSPGK |
| | | (SEQ ID NO: 101) |
| | LC | DIVMTQSQKFMSTSVGDRVSVTCKASQNVG |
| | | TNVAWYQQKLGQSPKTLIYSASYRYSGVPD |
| | | RFTGSGSGTDFTLTISNVQSEDLAEYFCQQ |
| | | YNSYPYTFGGGTKLEIKRADAAPTVSIFPP |
| | | SSEQLTSGGASVVCFLNNFYPKDINVKWKI |
| | | DGSERQNGVLNSWTDQDSKDSTYSMSSTLT |
| | | LTKDEYERHNSYTCEATHKTSTSPIVKSFN |
| | | RNEC |
| | | (SEQ ID NO: 102) |
| 06C11 | VH | QVTLKESGPGILQPSQTLSLTCSFSGFSLN |
| | | TYGMGVSWIRQPSGKGLEWLAHIYWDDDKR |
| | | YNPSLKSRLTISKDASNNRVFLKITSVDTA |
| | | DTATYYCAQRGYDDYWGYWGQGTLVTISA |
| | | (SEQ ID NO: 103) |
| | VL | DIVMTQSQKFMSTSVGDRVSVTCKASQNVG |
| | | TNVAWFQQKPGQSPKALIYSASYRYSGVPD |
| | | RFTGSGSGTDFILTISNVQSEDLAEYFCQQ |
| | | YNNYPLTFGAGTKLELK |
| | | (SEQ ID NO: 104) |
| | HC | QVTLKESGPGILQPSQTLSLTCSFSGFSLN |
| | | TYGMGVSWIRQPSGKGLEWLAHIYWDDDKR |
| | | YNPSLKSRLTISKDASNNRVFLKITSVDTA |
| | | DTATYYCAQRGYDDYWGYWGQGTLVTISAA |
| | | KTTPPSVYPLAPGSAAQTNSMVTLGCLVKG |
| | | YFPEPVTVTWNSGSLSSGVHTFPAVLQSDL |
| | | YTLSSSVTVPSSTWPSETVTCNVAHPASST |
| | | KVDKKIVPRDCGCKPCICTVPEVSSVFIFP |
| | | PKPKDVLTITITPKVTCVVVDISKDDPEVQ |
| | | FSWFVDDVEVHTAQTQPREEQFNSTFRSVS |
| | | ELPIMHQDWLNGKEFKCRVNSAAFPAPIEK |
| | | TISKTKGRPKAPQVYTIPPPKEQMAKDKVS |
| | | LTCMITDFFPEDITVEWQWNGQPAENYKNT |
| | | QPIMDTDGSYFVYSKLNVQKSNWEAGNTFT |
| | | CSVLHEGLHNHHTEKSLSHSPGK |
| | | (SEQ ID NO: 105) |
| | LC | DIVMTQSQKFMSTSVGDRVSVTCKASQNVG |
| | | TNVAWFQQKPGQSPKALIYSASYRYSGVPD |
| | | RFTGSGSGTDFILTISNVQSEDLAEYFCQQ |
| | | YNNYPLTFGAGTKLELKRADAAPTVSIFPP |
| | | SSEQLTSGGASVVCFLNNFYPKDINVKWKI |
| | | DGSERQNGVLNSWTDQDSKDSTYSMSSTLT |
| | | LTKDEYERHNSYTCEATHKTSTSPIVKSFN |
| | | RNEC |
| | | (SEQ ID NO: 106) |
| 08G01 | VH | EVLLQQSGPEVVKPGASVKIPCKASGYTFT |
| | | DYNMDWVKQSHGKSLEWIGEINPNNGGTFY |
| | | NQKFKGKATLTVDKSSSTAYMELRSLTSED |
| | | TAVYYCAREAITTVGAMDYWGQGTSVTVSS |
| | | (SEQ ID NO: 107) |
| | VL | DIQMTQSPASLSASVGETVTITCRASGNIH |
| | | NYLA+10WYQQKQGKSPQLLVYNAKTLADG |
| | | VPSRFSGSGSGTQYSLKINSLQPEDFGSYY |
| | | CQHFWSSPYTFGGGTKLEIK |
| | | (SEQ ID NO: 108) |
| | HC | EVLLQQSGPEVVKPGASVKIPCKASGYTFT |
| | | DYNMDWVKQSHGKSLEWIGEINPNNGGTFY |
| | | NQKFKGKATLTVDKSSSTAYMELRSLTSED |
| | | TAVYYCAREAITTVGAMDYWGQGTSVTVSS |
| | | AKTTPPSVYPLAPGCGDTTGSSVTLGCLVK |
| | | GYFPESVTVTWNSGSLSSSVHTFPALLQSG |
| | | LYTMSSSVTVPSSTWPSQTVTCSVAHPASS |
| | | TTVDKKLEPSGPISTINPCPPCKECHKCPA |
| | | PNLEGGPSVFIFPPNIKDVLMISLTPKVTC |
| | | VVVDVSEDDPDVQISWFVNNVEVHTAQTQT |
| | | HREDYNSTIR+10VVSTLPIQHQDWMSGKE |
| | | FKCKVNNKDLPSPIERTISKIKGLVRAPQV |
| | | YILPPPAEQLSRKDVSLTCLVVGFNPGDIS |
| | | VEWTSNGHTEENYKDTAPVLDSDGSYFIYS |
| | | KLNMKTSKWEKTDSFSCNVRHEGLKNYYLK |
| | | KTISRSPGK |
| | | (SEQ ID NO: 109) |
| | LC | DIQMTQSPASLSASVGETVTITCRASGNIH |
| | | NYLAWYQQKQGKSPQLLVYNAKTLADGVPS |
| | | RFSGSGSGTQYSLKINSLQPEDFGSYYCQH |
| | | FWSSPYTFGGGTKLEIKRADAAPTVSIFPP |
| | | SSEQLTSGGASVVCFLNNFYPKDINVKWKI |
| | | DGSERQNGVLNSWTDQDSKDSTYSMSSTLT |
| | | LTKDEYERHNSYTCEATHKTSTSPIVKSFN |
| | | RNEC |
| | | (SEQ ID NO: 110) |
| 14F11 | VH | QVTLKESGPGILQPSQTLSLTCSFSGFSLS |
| | | TYGMGVGWIRQPSGKGLEWLADIWWDDDKY |
| | | YNPSLKSRLTISKDTSSNEVFLKIAIVDTA |
| | | DTATYYCARRGHYSAMDYWGQGTSVTVSS |
| | | (SEQ ID NO: 111) |
| | VL | DIVMTQSQKFMSTSVGDRVSVTCKASQNVG |
| | | TNVAWYQQKPGQSPKALIYSPSYRYSGVPD |
| | | RFTGSGSGTDFTLTISNVQSEDLAEYFCQQ |
| | | YNSYPHTFGGGTKLEMK |
| | | (SEQ ID NO: 112) |
| | HC | QVTLKESGPGILQPSQTLSLTCSFSGFSLS |
| | | TYGMGVGWIRQPSGKGLEWLADIWWDDDKY |
| | | YNPSLKSRLTISKDTSSNEVFLKIAIVDTA |
| | | DTATYYCARRGHYSAMDYWGQGTSVTVSSA |
| | | KTTPPSVYPLAPGSAAQTNSMVTLGCLVKG |
| | | YFPEPVTVTWNSGSLSSGVHTFPAVLQSDL |
| | | YTLSSSVTVPSSTWPSETVTCNVAHPASST |
| | | KVDKKIVPRDCGCKPCICTVPEVSSVFIFP |
| | | PKPKDVLTITITPKVTCVVVDISKDDPEVQ |
| | | FSWFVDDVEVHTAQTQPREEQFNSTFRSVS |
| | | ELPIMHQDWLNGKEFKCRVNSAAFPAPIEK |
| | | TISKTKGRPKAPQVYTIPPPKEQMAKDKVS |
| | | LTCMITDFFPEDITVEWQWNGQPAENYKNT |
| | | QPIMDTDGSYFVYSKLNVQKSNWEAGNTFT |
| | | CSVLHEGLHNHHTEKSLSHSPGK |
| | | (SEQ ID NO: 113) |
| | LC | DIVMTQSQKFMSTSVGDRVSVTCKASQNVG |
| | | TNVAWYQQKPGQSPKALIYSPSYRYSGVPD |
| | | RFTGSGSGTDFTLTISNVQSEDLAEYFCQQ |
| | | YNSYPHTFGGGTKLEMKRADAAPTVSIFPP |
| | | SSEQLTSGGASVVCFLNNFYPKDINVKWKI |
| | | DGSERQNGVLNSWTDQDSKDSTYSMSSTLT |
| | | LTKDEYERHNSYTCEATHKTSTSPIVKSFN |
| | | RNEC |
| | | (SEQ ID NO: 114) |
| 17B11 | VH | QVTLKESGPGILQPSQTLSLTCSFSGFSLS |
| | | TSGMGVSWIRQPSGKGLEWLAHNDWDDDKR |
| | | YKSSLKSRLTISKDTSRNQVFLKITSVDTA |
| | | DTATYYCARRVGGLEGYFDYWGQGTTLTVS |
| | | S |
| | | (SEQ ID NO: 115) |
| | VL | DIVLTQSPASLAVSLGQRATISCRASQSVS |
| | | TSRFSYMHWFQQKPGQAPKLLIKYASNLES |
| | | GVPARFSGSGSGTDFTLNIHPVEGEDTATY |
| | | YCQHSWEIPYTFGGGTKLEIK |
| | | (SEQ ID NO: 116) |
| | HC | QVTLKESGPGILQPSQTLSLTCSFSGFSLS |
| | | TSGMGVSWIRQPSGKGLEWLAHNDWDDDKR |
| | | YKSSLKSRLTISKDTSRNQVFLKITSVDTA |
| | | DTATYYCARRVGGLEGYFDYWGQGTTLTVS |
| | | SAKTTPPSVYPLAPGSAAQTNSMVTLGCLV |
| | | KGYFPEPVTVTWNSGSLSSGVHTFPAVLQS |
| | | DLYTLSSSVTVPSSTWPSETVTCNVAHPAS |
| | | STKVDKKIVPRDCGCKPCICTVPEVSSVFI |
| | | FPPKPKDVLTITLTPKVTCVVVDISKDDPE |
| | | VQFSWFVDDVEVHTAQTQPREEQFNSTFRS |
| | | VSELPIMHQDWLNGKEFKCRVNSAAFPAPI |
| | | EKTISKTKGRPKAPQVYTIPPPKEQMAKDK |
| | | VSLTCMITDFFPEDITVEWQWNGQPAENYK |
| | | NTQPIMDTDGSYFVYSKLNVQKSNWEAGNT |
| | | FTCSVLHEGLHNHHTEKSLSHSPGK |
| | | (SEQ ID NO: 117) |
| | LC | DIVLTQSPASLAVSLGQRATISCRASQSVS |
| | | TSRFSYMHWFQQKPGQAPKLLIKYASNLES |
| | | GVPARFSGSGSGTDFTLNIHPVEGEDTATY |
| | | YCQHSWEIPYTFGGGTKLEIKRADAAPTVS |
| | | IFPPSSEQLTSGGASVVCFLNNFYPKDINV |
| | | KWKIDGSERQNGVLNSWTDQDSKDSTYSMS |
| | | STLTLTKDEYERHNSYTCEATHKTSTSPIV |
| | | KSFNRNEC |
| | | (SEQ ID NO: 118) |

In some embodiments, the GDF 15 inhibitor comprises AV-380. In some embodiments, the AV-380 is administered to the subject at a daily dose of between 1 mg/kg and 20 mg/kg, e.g. between 4 mg/kg and 16 mg/kg, e.g. between 8 mg/kg and 14 mg/kg per day.

### Visugromab

In some embodiments, the GDF15 inhibitor comprises an antibody molecule having one or more of (e.g., 1, 2, 3, 4, 5, or all of) a light chain complementary determining region 1 (LC CDR1), a light chain complementary determining region 2 (LC CDR2), a light chain complementary determining region 3 (LC CDR3), a heavy chain complementary determining region 1 (HC CDR1), a heavy chain complementary determining region 2 (HC CDR2), and a heavy chain complementary determining region 3 (HC CDR3) having amino acid sequences according to Table 6.

TABLE 6
CDRs of Visugromab

| HC CDR1 | HC CDR2 | HC CDR3 | LC CDR1 | LC CDR2 | LC CDR3 |
|---|---|---|---|---|---|
| GFSLS | IYWD | ARSSY | QNV | SAS | QQYNN |
| TSGMG | DDK | GAMDY | GTN | | FPYT |
| (SEQ ID | (SEQ ID | (SEQ ID | (SEQ ID | | (SEQ ID |
| NO: | NO: | NO: | NO: | | NO: |
| 62 | 55) | 119) | 88) | | 120) |

In some embodiments, the GDF15 inhibitor comprises an antibody molecule having a heavy chain variable region (VH) comprising a VH amino acid sequence according to Table 7, or a sequence having at least 70%, 75%, 80%, 85%, 90%, 95%, 97%, 98%, or 99% identity thereto. In some embodiments, the GDF15 inhibitor comprises an antibody molecule having a light chain variable region (VL) comprising a VL amino acid sequence according to Table 7, or a sequence having at least 70%, 75%, 80%, 85%, 90%, 95%, 97%, 98%, or 99% identity thereto. In some embodiments, the GDF15 inhibitor comprises an antibody molecule having a heavy chain (HC) comprising a HC amino acid sequence according to Table 7, or a sequence having at least 70%, 75%, 80%, 85%, 90%, 95%, 97%, 98%, or 99% identity thereto. In some embodiments, the GDF15 inhibitor comprises an antibody molecule having a light chain (LC) comprising a LC amino acid sequence according to Table 7, or a sequence having at least 70%, 75%, 80%, 85%, 90%, 95%, 97%, 98%, or 99% identity thereto.

TABLE 7
VH, VL, HC, and LC of Visugromab

| | |
|---|---|
| VH | QITLKESGPTLVKPTQTLTLTCTFSGFSLSTSGMG |
| | VSWIRQPPGKGLEWLAHIYWDDDKRYNPTLKSRLT |
| | ITKDPSKNQVVLTMTNMDPVDTATYYCARSSYGAM |
| | DYWGQGTLVTVSS |
| | (SEQ ID NO: 121) |
| VL | DIVLTQSPSFLSASVGDRVTITCKASQNVGTNVAW |
| | FQQKPGKSPKALIYSASYRYSGVPDRFTGSGSGTE |
| | FTLTISSLQPEDFAAYFCQQYNNFPYTFGGGTKLE |
| | IKR |
| | (SEQ ID NO: 122) |
| HC | QITLKESGPTLVKPTQTLTLTCTFSGFSLSTSGMG |
| | VSWIRQPPGKGLEWLAHIYWDDDKRYNPTLKSRLT |
| | ITKDPSKNQVVLTMTNMDPVDTATYYCARSSYGAM |
| | DYWGQGTLVTVSSASTKGPSVFPLAPCSRSTSEST |
| | AALGCLVKDYFPEPVTVSWNSGALTSGVHTFPAVL |
| | QSSGLYSLSSVVTVPSSSLGTKTYTCNVDHKPSNT |
| | KVDKRVESKYGPPCPPCPAPEFLGGPSVFLFPPKP |
| | KDTLMISRTPEVTCVVVDVSQEDPEVQFNWYVDGV |
| | EVHNAKTKPREEQFNSTYRVVSVLTVLHQDWLNGK |
| | EYKCKVSNKGLPSSIEKTISKAKGQPREPQVYTLP |
| | PSQEEMTKNQVSLTCLVKGFYPSDIAVEWESNGQ |
| | PENNYKTTPPVLDSDGSFFLYSRLTVDKSRWQEGN |
| | VFSCSVMHEALHNHYTQKSLSLSLG |
| | (SEQ ID NO: 123) |
| LC | DIVLTQSPSFLSASVGDRVTITCKASQNVGTNVAW |
| | FQQKPGKSPKALIYSASYRYSGVPDRFTGSGSGTE |
| | FTLTISSLQPEDFAAYFCQQYNNFPYTFGGGTKLE |
| | IKRTVAAPSVFIFPPSDEQLKSGTASVVCLLNNFY |
| | PREAKVQWKVDNALQSGNSQESVTEQDSKDSTYSL |
| | SSTLTLSKADYEKHKVYACEVTHQGLSSPVTKSFN |
| | RGEC |
| | (SEQ ID NO: 124) |

In some embodiments, the GDF 15 inhibitor comprises Visugromab. In some embodiments, the Visugromab is administered to the subject at a daily dose of between 0.3 mg/kg and 20 mg/kg, e.g. between 2 mg/kg and 15 mg/kg, e.g. between 8 mg/kg and 12 mg/kg per day.

### Methods of Treatment

The present disclosure provides, for instance, methods of treating cancer. In some embodiments, the cancer is a solid tumor. In some embodiments, the cancer is chosen from chosen from breast cancer, ovarian cancer, pancreatic cancer, fallopian tube cancer, primary peritoneal cancer, or prostate cancer.

In some embodiments, at the time of treatment, the subject has, or is identified as having, stable disease or progressive disease. The therapies can be administered during periods of active disorder, or during a period of remission or less active disease. The first therapy can be administered before the second therapy, concurrently with the treatment, post-treatment, or during remission of the disorder. In some embodiments, after the treatment described herein, the subject experiences a partial response, a complete response and/or prolonged response or delayed relapse (e.g., compared to the expected course of disease when treated without the treatment described herein).

In some embodiments, the subject is or is identified as being, at risk of developing resistance to, or at risk of being refractory to, a DDR inhibitor. In some embodiments, the subject is initially responsive to the DDR inhibitor, but then becomes resistant or refractory during the course of treatment with the DDR inhibitor. In some embodiments, the subject was resistant or refractory at the outset of DDR inhibitor treatment.

In some embodiments, the subject expresses GDF15, e.g., expresses a detectable amount of GDF15. In some embodiments, the tumor expresses GDF15, e.g., expresses a detectable amount of GDF15.

In some embodiments, when administered in combination, one or both of the DDR inhibitor and the GDF15 inhibitor, may be administered in an amount or dose that is higher, lower or the same than the amount or dosage of each agent used individually, e.g., as a monotherapy. In some embodiments, when administered in combination, one or both of the DDR inhibitor and the GDF15 inhibitor, may be administered with a duration that is longer, shorter, or the same than the duration of each agent used individually, e.g., as a monotherapy.

The administration of the compositions described herein may be carried out in any convenient manner, including by aerosol inhalation, injection, ingestion, transfusion, implantation or transplantation. The compositions described herein may be administered to a patient orally, trans-arterially, subcutaneously, intradermally, intratumorally, intranodally, intramedullary, intramuscularly, by intravenous (i.v.) injection, or intraperitoneally.

In some embodiments, the combination therapies described herein result in a synergistic effect. In some embodiments, the combination therapies described herein result in an additive effect.

In some embodiments, two (or more) different treatments are delivered to the subject in combination, during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

### OTHER EMBODIMENTS

Specific compositions and methods for the treatment of cancer have been described. The scope of the invention should be defined by the claims. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the Applicant's information and belief. The Applicant makes no admission about the correctness of the contents or dates of these documents. Should there be a discrepancy between a date provided in this specification and the actual publication date, then the actual publication date shall control. Should there be a discrepancy between the scientific or technical teaching of a previous patent or publication and this specification, then the teaching of this specification and these claims shall control.

The foregoing written specification is considered sufficient to enable one skilled in the biomedical art to practice the present aspects and embodiments. The present aspects and embodiments are not to be limited in scope by examples provided, since the examples are intended as a single illustration of one aspect and other functionally equivalent embodiments are within the scope of the disclosure. Various modifications besides those shown and described herein will become apparent to those skilled in the biomedical art from the foregoing description and fall within the scope of the appended claims. The advantages and objects described herein are not necessarily encompassed by each embodiment. Those skilled in the biomedical art will recognize or be able to ascertain using no more than routine experimentation, many equivalents to the specific embodiments described herein. Such equivalents are intended to be encompassed by these claims.

1. A method of treating cancer in a subject, the method comprising:
administering to the subject an effective amount of a DNA damage and repair (DDR) inhibitor, wherein the subject receives, in combination, a GDF15 inhibitor,
thereby treating the cancer in the subject.

2. (canceled)

3. A method of inhibiting resistance to a DDR inhibitor in a subject, the method comprising administering to the subject a GDF15 inhibitor, thereby inhibiting resistance to the DDR inhibitor in the subject.

4. A kit comprising:
a DDR inhibitor or a nucleic acid encoding the DDR inhibitor; and
a GDF15 inhibitor or a nucleic acid encoding the GDF15 inhibitor.

5. The method of claim 1, wherein the DDR inhibitor comprises:
(i) an ATR inhibitor or a PARP inhibitor; and/or
(ii) a small molecule, an antibody molecule, a nucleic acid, or a polypeptide.

6. (canceled)

7. The method of claim 1, wherein the DDR inhibitor comprises ceralasertib, camonsertib, elimusertib, or berzosertib.

8. The method of claim 1, wherein the DDR inhibitor comprises ceralasertib, camonsertib, or elimusertib, and is administered daily.

9.-11. (canceled)

12. The method of claim 1, wherein the GDF15 inhibitor comprises a small molecule, an antibody molecule, a nucleic acid, or a polypeptide.

13. The method of claim 1, wherein the GDF15 inhibitor comprises an antibody molecule having a light chain complementarity determining region 1 (LC CDR1), a light chain complementarity determining region 2 (LC CDR2), a light chain complementarity determining region 3 (LC CDR3), a heavy chain complementarity determining region 1 (HC CDR1), a heavy chain complementarity determining region 2 (HC CDR2), and a heavy chain complementarity determining region 3 (HC CDR3) having amino acid sequences according to Table 1, Table 3, Table 4, or Table 6.

14. The method or kit of claim 1, wherein the GDF15 inhibitor comprises an antibody molecule having a light chain variable region (VL) comprising an amino acid sequence according to Table 2, Table 5, or Table 7, or a sequence having at least 90%, 95%, 97%, 98%, or 99% identity thereto, and/or a heavy chain variable region (VH) comprising an amino acid sequence according to Table 2, Table 5, or Table 7, or a sequence having at least 90%, 95%, 97%, 98%, or 99% identity thereto.

15. (canceled)

16. The method of claim 1, wherein the GDF15 inhibitor comprises AV-380, ponsegromab, or visugromab.

17. The method of claim 1, wherein the DDR inhibitor and/or the GDF15 inhibitor is administered orally, parenterally, intravenously, or topically.

18. (canceled)

19. The method of claim 1, wherein:
the DDR inhibitor and the GDF15 inhibitor are administered simultaneously;
the DDR inhibitor is administered before the GDF15 inhibitor; or
the GDF15 inhibitor is administered before the DDR inhibitor.

20. The method of claim 1, wherein the subject comprises a mutation in BRCA1 or BRCA2.

21. The method of claim 1, wherein the subject:
(i) is resistant to or refractory to a DDR inhibitor; or
(ii) is, or is identified as being, at risk of developing resistance to, or at risk of being refractory to, a DDR inhibitor.

22. (canceled)

23. The method of claim 1, wherein following administration of the GDF15 inhibitor, the subject:
(i) does not develop resistance to the DDR inhibitor, or wherein resistance to the DDR inhibitor is delayed or decreased; and/or
(ii) displays increased sensitivity to the DDR inhibitor.

24. (canceled)

25. The method of claim 1, wherein the subject has a solid tumor.

26. The method of claim 1, wherein the cancer is chosen from breast cancer, ovarian cancer, pancreatic cancer, fallopian tube cancer, primary peritoneal cancer, or prostate cancer.

27. The method of claim 1, wherein the subject has a liquid tumor.

28. The method of claim 1, wherein the cancer is a leukemia or lymphoma.

29. The method of claim 1, wherein the subject is a human.


SEQUENCE LISTING (124 sequences)

SEQ ID NO: 1 - protein, 10 aa, synthetic construct
GYTFSSYNID

SEQ ID NO: 2 - protein, 17 aa, synthetic construct
GINPIFGTAFYNQKFQG

SEQ ID NO: 3 - protein, 11 aa, synthetic construct
EAITTVGAMDH

SEQ ID NO: 4 - protein, 11 aa, synthetic construct
RTSQSVHNYLA

SEQ ID NO: 5 - protein, 7 aa, synthetic construct
DASTRAD

SEQ ID NO: 6 - protein, 9 aa, synthetic construct
QQFWSWPWT

SEQ ID NO: 7 - protein, 120 aa, synthetic construct
QVQLVQSGAEVKKPGSSVKVSCKASGYTFSSYNIDWVRQAPGQGLEWMGGINPIFGTAFYNQKFQGRVTITADESTSTAYMELSSLRSEDTAVYYCAREAITTVGAMDHWGQGTLVTVSS

SEQ ID NO: 8 - protein, 107 aa, synthetic construct
EIVLTQSPATLSLSPGERATLSCRTSQSVHNYLAWYQQKPGQAPRLLIYDASTRADGIPARFSGSGSGTDFTLTISSLEPEDFAVYYCQQFWSWPWTFGQGTKVEIK

SEQ ID NO: 9 - protein, 449 aa, synthetic construct
QVQLVQSGAEVKKPGSSVKVSCKASGYTFSSYNIDWVRQAPGQGLEWMGGINPIFGTAFYNQKFQGRVTITADESTSTAYMELSSLRSEDTAVYYCAREAITTVGAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPATLSLSPGERATLSCRTSQSVHNYLAWYQQKPGQAPRLLIYDASTRADGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
DYNMD

SEQ ID NO: 12 - protein, 17 aa, synthetic construct
QINPNNGGIFFNQKFKG

SEQ ID NO: 13 - protein, 11 aa, synthetic construct
EAITTVGAMDY

SEQ ID NO: 14 - protein, 5 aa, synthetic construct
SYWIH

SEQ ID NO: 15 - protein, 17 aa, synthetic construct
DINPSNGRSKYNEKFKN

SEQ ID NO: 16 - protein, 9 aa, synthetic construct
EVLDGAMDY

SEQ ID NO: 17 - protein, 7 aa, synthetic construct
TYGMGVT

SEQ ID NO: 18 - protein, 16 aa, synthetic construct
HIYWDDDKRYNPSLKS

SEQ ID NO: 19 - protein, 9 aa, synthetic construct
TGYSNLFAY

SEQ ID NO: 20 - protein, 7 aa, synthetic construct
TYGMGVS

SEQ ID NO: 21 - protein, 9 aa, synthetic construct
RGYDDYWGY

SEQ ID NO: 22 - protein, 17 aa, synthetic construct
EINPNNGGTFYNQKFKG

SEQ ID NO: 23 - protein, 7 aa, synthetic construct
TYGMGVG

SEQ ID NO: 24 - protein, 16 aa, synthetic construct
DIWWDDDKYYNPSLKS

SEQ ID NO: 25 - protein, 9 aa, synthetic construct
RGHYSAMDY

SEQ ID NO: 26 - protein, 7 aa, synthetic construct
TSGMGVS

SEQ ID NO: 27 - protein, 16 aa, synthetic construct
HNDWDDDKRYKSSLKS

SEQ ID NO: 28 - protein, 11 aa, synthetic construct
RVGGLEGYFDY

SEQ ID NO: 29 - protein, 17 aa, synthetic construct
QINPNNGGIFFNQKFQG

SEQ ID NO: 30 - protein, 17 aa, synthetic construct
QINPYNHLIFFNQKFQG

SEQ ID NO: 31 - protein, 17 aa, synthetic construct
QINPNNGLIFFNQKFQG

SEQ ID NO: 32 - protein, 17 aa, synthetic construct
QINPNNGLIFFNQKFKG

SEQ ID NO: 33 - protein, 17 aa, synthetic construct
QINPYNHLIFFNQKFKG

SEQ ID NO: 34 - protein, 16 aa, synthetic construct
HIYWDDDKRYNPSLKT

SEQ ID NO: 35 - protein, 7 aa, synthetic construct
GYTFTDY

SEQ ID NO: 36 - protein, 6 aa, synthetic construct
NPNNGG

SEQ ID NO: 37 - protein, 7 aa, synthetic construct
GYTFTSY

SEQ ID NO: 38 - protein, 6 aa, synthetic construct
NPSNGR

SEQ ID NO: 39 - protein, 9 aa, synthetic construct
GFSLSTYGM

SEQ ID NO: 40 - protein, 5 aa, synthetic construct
YWDDD

SEQ ID NO: 41 - protein, 9 aa, synthetic construct
GFSLNTYGM

SEQ ID NO: 42 - protein, 5 aa, synthetic construct
WWDDD

SEQ ID NO: 43 - protein, 9 aa, synthetic construct
GFSLSTSGM

SEQ ID NO: 44 - protein, 5 aa, synthetic construct
DWDDD

SEQ ID NO: 45 - protein, 7 aa, synthetic construct
GYTFSDY

SEQ ID NO: 46 - protein, 6 aa, synthetic construct
NPYNHL

SEQ ID NO: 47 - protein, 6 aa, synthetic construct
NPNNGL

SEQ ID NO: 48 - protein, 8 aa, synthetic construct
GYTFTDYN

SEQ ID NO: 49 - protein, 8 aa, synthetic construct
INPNNGGI

SEQ ID NO: 50 - protein, 13 aa, synthetic construct
AREAITTVGAMDY

SEQ ID NO: 51 - protein, 8 aa, synthetic construct
GYTFTSYW

SEQ ID NO: 52 - protein, 8 aa, synthetic construct
INPSNGRS

SEQ ID NO: 53 - protein, 11 aa, synthetic construct
AREVLDGAMDY

SEQ ID NO: 54 - protein, 10 aa, synthetic construct
GFSLSTYGMG

SEQ ID NO: 55 - protein, 7 aa, synthetic construct
IYWDDDK

SEQ ID NO: 56 - protein, 11 aa, synthetic construct
AQTGYSNLFAY

SEQ ID NO: 57 - protein, 10 aa, synthetic construct
GFSLNTYGMG

SEQ ID NO: 58 - protein, 11 aa, synthetic construct
AQRGYDDYWGY

SEQ ID NO: 59 - protein, 8 aa, synthetic construct
INPNNGGT

SEQ ID NO: 60 - protein, 7 aa, synthetic construct
IWWDDDK

SEQ ID NO: 61 - protein, 11 aa, synthetic construct
ARRGHYSAMDY

SEQ ID NO: 62 - protein, 10 aa, synthetic construct
GFSLSTSGMG

SEQ ID NO: 63 - protein, 7 aa, synthetic construct
NDWDDDK

SEQ ID NO: 64 - protein, 13 aa, synthetic construct
ARRVGGLEGYFDY

SEQ ID NO: 65 - protein, 8 aa, synthetic construct
GYTFSDYN

SEQ ID NO: 66 - protein, 8 aa, synthetic construct
INPYNHLI

SEQ ID NO: 67 - protein, 8 aa, synthetic construct
INPNNGLI

SEQ ID NO: 68 - protein, 11 aa, synthetic construct
RTSENLHNYLA

SEQ ID NO: 69 - protein, 7 aa, synthetic construct
DAKTLAD

SEQ ID NO: 70 - protein, 9 aa, synthetic construct
QHFWSSPYT

SEQ ID NO: 71 - protein, 15 aa, synthetic construct
RASESVDNYGISFMN

SEQ ID NO: 72 - protein, 7 aa, synthetic construct
AASNQGS

SEQ ID NO: 73 - protein, 9 aa, synthetic construct
QQSKEVPWT

SEQ ID NO: 74 - protein, 11 aa, synthetic construct
KASQNVGTNVA

SEQ ID NO: 75 - protein, 7 aa, synthetic construct
SASYRYS

SEQ ID NO: 76 - protein, 9 aa, synthetic construct
QQYNSYPYT

SEQ ID NO: 77 - protein, 9 aa, synthetic construct
QQYNNYPLT

SEQ ID NO: 78 - protein, 11 aa, synthetic construct
RASGNIHNYLA

SEQ ID NO: 79 - protein, 7 aa, synthetic construct
NAKTLAD

SEQ ID NO: 80 - protein, 7 aa, synthetic construct
SPSYRYS

SEQ ID NO: 81 - protein, 9 aa, synthetic construct
QQYNSYPHT

SEQ ID NO: 82 - protein, 15 aa, synthetic construct
RASQSVSTSRFSYMH

SEQ ID NO: 83 - protein, 7 aa, synthetic construct
YASNLES

SEQ ID NO: 84 - protein, 9 aa, synthetic construct
QHSWEIPYT

SEQ ID NO: 85 - protein, 9 aa, synthetic construct
QHFWSDPYT

SEQ ID NO: 86 - protein, 6 aa, synthetic construct
ENLHNY

SEQ ID NO: 87 - protein, 10 aa, synthetic construct
ESVDNYGISF

SEQ ID NO: 88 - protein, 6 aa, synthetic construct
QNVGTN

SEQ ID NO: 89 - protein, 6 aa, synthetic construct
GNIHNY

SEQ ID NO: 90 - protein, 10 aa, synthetic construct
QSVSTSRFSY

SEQ ID NO: 91 - protein, 120 aa, synthetic construct
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSS

SEQ ID NO: 92 - protein, 107 aa, synthetic construct
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIK

SEQ ID NO: 93 - protein, 444 aa, synthetic construct
EVLLQQSGPELVKPGASVKIPCKASGYTFTDYNMDWVKQSHGKSLEWIGQINPNNGGIFFNQKFKGKATLTVDKSSNTAFMEVRSLTSEDTAVYYCAREAITTVGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 94 - protein, 214 aa, synthetic construct
DIQMTQSPASLSASVGETVTITCRTSENLHNYLAWYQQKQGKSPQLLVYDAKTLADGVPSRFSGSGSGTQYSLKINSLQPEDFGSYYCQHFWSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 95 - protein, 118 aa, synthetic construct
QVQLQQPGAELVKPGASVKLSCKASGYTFTSYWIHWVNQRPGQGLEWIGDINPSNGRSKYNEKFKNKATMTADKSSNTAYMQLSSLTSEDSAVYYCAREVLDGAMDYWGQGTSVTVSS

SEQ ID NO: 96 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGSKLEIK

SEQ ID NO: 97 - protein, 442 aa, synthetic construct
QVQLQQPGAELVKPGASVKLSCKASGYTFTSYWIHWVNQRPGQGLEWIGDINPSNGRSKYNEKFKNKATMTADKSSNTAYMQLSSLTSEDSAVYYCAREVLDGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 98 - protein, 218 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGSKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 99 - protein, 119 aa, synthetic construct
QVTLKESGPGILQPSQTLSLTCSFSGFSLSTYGMGVTWIRQPSGKGLEWLAHIYWDDDKRYNPSLKSRLTISKDTSNNQVFLKITSVDTADTATYYCAQTGYSNLFAYWGQGTLVTVSA

SEQ ID NO: 100 - protein, 107 aa, synthetic construct
DIVMTQSQKFMSTSVGDRVSVTCKASQNVGTNVAWYQQKLGQSPKTLIYSASYRYSGVPDRFTGSGSGTDFTLTISNVQSEDLAEYFCQQYNSYPYTFGGGTKLEIK

SEQ ID NO: 101 - protein, 443 aa, synthetic construct
QVTLKESGPGILQPSQTLSLTCSFSGFSLSTYGMGVTWIRQPSGKGLEWLAHIYWDDDKRYNPSLKSRLTISKDTSNNQVFLKITSVDTADTATYYCAQTGYSNLFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITITPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 102 - protein, 214 aa, synthetic construct
DIVMTQSQKFMSTSVGDRVSVTCKASQNVGTNVAWYQQKLGQSPKTLIYSASYRYSGVPDRFTGSGSGTDFTLTISNVQSEDLAEYFCQQYNSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 103 - protein, 119 aa, synthetic construct
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISA

SEQ ID NO: 104 - protein, 107 aa, synthetic construct
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELK

SEQ ID NO: 105 - protein, 443 aa, synthetic construct
QVTLKESGPGILQPSQTLSLTCSFSGFSLNTYGMGVSWIRQPSGKGLEWLAHIYWDDDKRYNPSLKSRLTISKDASNNRVFLKITSVDTADTATYYCAQRGYDDYWGYWGQGTLVTISAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITITPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 106 - protein, 214 aa, synthetic construct
DIVMTQSQKFMSTSVGDRVSVTCKASQNVGTNVAWFQQKPGQSPKALIYSASYRYSGVPDRFTGSGSGTDFILTISNVQSEDLAEYFCQQYNNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 107 - protein, 120 aa, synthetic construct
EVLLQQSGPEVVKPGASVKIPCKASGYTFTDYNMDWVKQSHGKSLEWIGEINPNNGGTFYNQKFKGKATLTVDKSSSTAYMELRSLTSEDTAVYYCAREAITTVGAMDYWGQGTSVTVSS

SEQ ID NO: 108 - protein, 107 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSSPYTFGGGTKLEIK

SEQ ID NO: 109 - protein, 456 aa, synthetic construct
EVLLQQSGPEVVKPGASVKIPCKASGYTFTDYNMDWVKQSHGKSLEWIGEINPNNGGTFYNQKFKGKATLTVDKSSSTAYMELRSLTSEDTAVYYCAREAITTVGAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 110 - protein, 214 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 111 - protein, 119 aa, synthetic construct
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSS

SEQ ID NO: 112 - protein, 107 aa, synthetic construct
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMK

SEQ ID NO: 113 - protein, 443 aa, synthetic construct
QVTLKESGPGILQPSQTLSLTCSFSGFSLSTYGMGVGWIRQPSGKGLEWLADIWWDDDKYYNPSLKSRLTISKDTSSNEVFLKIAIVDTADTATYYCARRGHYSAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITITPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 114 - protein, 214 aa, synthetic construct
DIVMTQSQKFMSTSVGDRVSVTCKASQNVGTNVAWYQQKPGQSPKALIYSPSYRYSGVPDRFTGSGSGTDFTLTISNVQSEDLAEYFCQQYNSYPHTFGGGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 115 - protein, 121 aa, synthetic construct
QVTLKESGPGILQPSQTLSLTCSFSGFSLSTSGMGVSWIRQPSGKGLEWLAHNDWDDDKRYKSSLKSRLTISKDTSRNQVFLKITSVDTADTATYYCARRVGGLEGYFDYWGQGTTLTVSS

SEQ ID NO: 116 - protein, 111 aa, synthetic construct
DIVLTQSPASLAVSLGQRATISCRASQSVSTSRFSYMHWFQQKPGQAPKLLIKYASNLESGVPARFSGSGSGTDFTLNIHPVEGEDTATYYCQHSWEIPYTFGGGTKLEIK

SEQ ID NO: 117 - protein, 445 aa, synthetic construct
QVTLKESGPGILQPSQTLSLTCSFSGFSLSTSGMGVSWIRQPSGKGLEWLAHNDWDDDKRYKSSLKSRLTISKDTSRNQVFLKITSVDTADTATYYCARRVGGLEGYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 118 - protein, 218 aa, synthetic construct
DIVLTQSPASLAVSLGQRATISCRASQSVSTSRFSYMHWFQQKPGQAPKLLIKYASNLESGVPARFSGSGSGTDFTLNIHPVEGEDTATYYCQHSWEIPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 119 - protein, 10 aa, synthetic construct
ARSSYGAMDY

SEQ ID NO: 120 - protein, 9 aa, synthetic construct
QQYNNFPYT

SEQ ID NO: 121 - protein, 118 aa, synthetic construct
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSS

SEQ ID NO: 122 - protein, 108 aa, synthetic construct
DIVLTQSPSFLSASVGDRVTITCKASQNVGTNVAWFQQKPGKSPKALIYSASYRYSGVPDRFTGSGSGTEFTLTISSLQPEDFAAYFCQQYNNFPYTFGGGTKLEIKR

SEQ ID NO: 123 - protein, 444 aa, synthetic construct
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 124 - protein, 214 aa, synthetic construct
DIVLTQSPSFLSASVGDRVTITCKASQNVGTNVAWFQQKPGKSPKALIYSASYRYSGVPDRFTGSGSGTEFTLTISSLQPEDFAAYFCQQYNNFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC